(19) United States
(12) Patent Application Publication
(10) Pub. No.: US 2024/0270855 A1
(43) Pub. Date: Aug. 15, 2024

(54) NEURO-MESENCHYME UNITS CONTROL ILC2 AND OBESITY VIA A BRAIN-ADIPOSE CIRCUIT

(71) Applicant: Fundação D. Anna de Sommer Champalimaud e Dr. Carlos Montez Champalimaud, Lisboa (PT)

(72) Inventors: José Henrique Veiga Fernandes, Oeiras (PT); Ana Filipa Ribeiro Cardoso, Lisboa (PT)

(73) Assignee: Fundação D. Anna de Sommer Champalimaud e Dr. Carlos Montez Champalimaud, Lisboa (PT)

(21) Appl. No.: 18/565,581

(22) PCT Filed: Jun. 2, 2022
(86) PCT No.: PCT/IB2022/000309

§ 371 Date: Nov. 30, 2023

Related U.S. Application Data
(60) Provisional application No. 63/196,266, filed on Jun. 3, 2021.

(51) Int. Cl. C07K 16/28; A61K 31/137; A61K 31/27; A61K 31/44; A61K 31/4704; A61K 31/4706; A61K 31/538; A61K 31/55; A61K 38/17; A61K 38/18; A61K 39/395; A61P 3/04

(52) U.S. Cl. CPC C07K 16/2863; A61K 31/137; A61K 31/27; A61K 31/44; A61K 31/4704; A61K 31/4706; A61K 31/538; A61K 31/55; A61K 38/179; A61K 38/185; A61K 39/3955; A61P 3/04

(57) ABSTRACT

The present disclosure provides a neuro-mesenchyme signaling axis that controls group 2 innate lymphoid cells (ILC2s), adipose tissue physiology, metabolism, and obesity. This signaling axis includes ILC2s with rearranged during transfection (RET) receptor, mesenchymal stromal cells (MSCs) with beta-2 adrenergic receptor (ADRB2), and high-order brain areas including the paraventricular nucleus of the hypothalamus (PVH).

### BACKGROUND

Sympathetic neurons interact with adipocytes and immune cells contribute to adipose tissue biology. Interactions between the nervous and immune systems have recently emerged as major regulators of host defense and inflammation¹⁻⁴. Nevertheless, whether neuronal and immune cells cooperate in brain-body axes to orchestrate metabolism is unknown.

### SUMMARY

The present disclosure is based on the discovery of a neuro-mesenchyme signaling axis that controls Group 2 innate lymphoid cells (ILC2s), adipose tissue physiology, metabolism, and obesity via a brain-adipose tissue circuit. Sympathetic neurons in adipose tissue act on neighboring adipose mesenchymal stromal cells (MSCs) via the beta-2 adrenergic receptor (ADRB2) to control the expression of glial-derived neurotrophic factor (GDNF) and the activity of gonadal adipose tissue (GAT) ILC2s. The neuro-mesenchyme signaling axis also modulates gonadal adipose tissue (GAT) ILC2s by connecting to high-order brain areas, including the paraventricular nucleus of the hypothalamus (PVH).

Accordingly, the present disclosure provides methods for manipulating ILC2 signaling that leads to energy expenditure, insulin resistance, and propensity to obesity.

In some aspects, the present disclosure provides methods for increasing activity or proliferation of Group 2 innate lymphoid cells (ILC2s) including contacting ILC2s with a rearranged during transfection (RET) agonist. In some aspects, the present disclosure provides methods for increasing activity of ILC2s including contacting mesenchymal stromal cells (MSCs) with a beta-2-adrenergic receptor (ADRB2) agonist.

In some aspects, the present disclosure provides methods for increasing production of interleukin-5 (IL-5), interleukin-13 (IL-13), and/or Met-enkephalin (Met-Enk) by ILC2s including contacting adipose ILC2s with a RET agonist and/or contacting MSCs with an ADRB2 agonist.

In some aspects, the present disclosure provides methods for decreasing susceptibility to obesity and/or increasing adipose homeostasis including: (a) administering to a subject a RET agonist that contacts ILC2s in adipose tissue, (b) administering to the subject an ADRB2 agonist that contacts MSCs in adipose tissue, or (c) a combination thereof. In some embodiments, increased adipose homeostasis is increased glucose tolerance and/or decreased gonadal adipose tissue (GAT) fat mass.

In some aspects, the present disclosure provides methods of treating a disorder associated with decreased ILC2 activity or proliferation including: (a) administering to a subject a RET agonist that contacts ILC2s in adipose tissue, (b) administering to the subject an ADRB2 agonist that contacts MSCs in adipose tissue, or a combination thereof.

In some aspects, the present disclosure provides methods of treating cold exposure including: (a) administering to a subject a RET agonist that contacts ILC2s, (b) administering to the subject an ADRB2 agonist, or (c) a combination thereof. In some embodiments, administering the RET agonist and/or the ADRB2 agonist increases the body temperature of the subject.

In some embodiments, the RET agonist includes: (1) a combination of a soluble GDNF Family binding Receptor alpha (GFRα) and a GFRα ligand (GFL) or an analog or mimetic thereof; or (2) an antibody that specifically binds to RET and increases RET tyrosine kinase activity or an antigen-binding fragment thereof.

In some embodiments, the combination of a soluble GFRα and GFRα ligand or an analog mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the ADRB2 agonist is clenbuterol, bitolterol, fenoterol, isoproterenol, levalbuterol, metaproterenol, pirbuterol, procaterol, ritodrine, albuterol, terbutaline, albuterol, aformoterol, bambuterol, formoterol, salmeterol, abediterol, carmoterol, indacaterol, olodaterol, vailanterol, isoxsuprine, mabuterol, zilpaterol, or a combination thereof.

In some aspects, the present disclosure provides methods of treating a disorder associated with increased ILC2 activity or proliferation including: (a) administering to a subject a RET antagonist that contacts ILC2s in adipose tissue, (b) administering to the subject an ADRB2 antagonist that contacts MSCs in adipose tissue, or (c) a combination of (a) and (b). In some embodiments, the disorder is hypothermia, cachexia, allergy, helminth infection, allergic asthma, atopic dermatitis, intestinal inflammatory disease, or a combination thereof.

In some embodiments, the RET antagonist is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib. In some embodiments, the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN). In some embodiments, the inhibitory nucleic acid molecule is a sRNA, shRNA, or antisense nucleic acid molecule.

In some embodiments, the ADRB2 antagonist is butoxamine, ICI-118,551, propranolol, oxprenolol, penbutolol, pindolol, sotalol, timolol, bucindolol, carteolol, carvedilol, labetalol, nadolol, or a combination thereof.

In some embodiments, the contacting is in vitro. In some embodiments, the contacting is in vivo. In some embodiments, the RET agonist, ADRB2 agonist, RET antagonist, and/or ADRB2 antagonist is administered to a subject. In some embodiments, the subject is a human.

In some embodiments, the ILC2s and/or the MSCs are in adipose tissue or derived from adipose tissue. In some embodiments, the adipose tissue is gonadal adipose tissue (GAT).

### BRIEF DESCRIPTION OF THE DRAWINGS

FIGS. 1A-1L. Sympathetic-mesenchyme interactions control ILC2 in the gonadal adipose tissue (GAT). FIG. 1A shows GAT, stained for sympathetic nerve fibers with tyrosine hydroxylase (TH, upper panel) and endothelial cells (CD31, lower panel). Scale bar: 300 μm. FIG. 1B shows ILC2 function after 6-hydroxydopamine (6-OHDA) administration. n=5. FIG. 1C shows ILC2 function after pegylated diphtheria toxin (PegDT)-treatment. R26/DTR^{fl} is diphtheria toxin receptor (DTR) inserted into a ROSA26 locus in a floxed mouse (Fl); R26/DTR^{Th} is DTR inserted into a ROSA26 locus in sympathetic nerve cells (Th). n=4. FIG. 1D shows ILC2 function after clenbuterol administration. n=5. FIG. 1E shows ILC2 function after clozapine N-oxide (CNO) administration. R26/3D^{fl} is DREADD-carrying adeno-associated virus inserted into a ROSA26 locus in a floxed mouse where n=5; R26/3D^{ΔTh} is DREADD-carrying adeno-associated virus inserted into a ROSA26 locus in a TH knock-out mouse where n=4. FIG. 1F shows GAT ILC2 activity in cells with wild-type beta-2 adrenergic receptor (Adrb2^{WT}) where n=7 and cells with ADRB2 deleted from lymphoid cells (Adrb2^{ΔIl7ra}) where n=8. FIG. 1G shows ILC2 function after 6-OHDA administration. Adrb2w n=13; Adrb2ΔIl7ra n=15. FIG. 1H shows GAT cell populations. n=6. PDGFRA+MSC is platelet derived growth factor receptor alpha positive mesenchymal stem cells; PDGFRA-MSC is platelet derived growth factor receptor alpha negative mesenchymal stem cells FIG. 11 shows sympathetic nerve fibers (TH, green), glial cells (GFAP, red), and cell nuclei (DAPI, blue). Scale bar: 50 μm. FIG. 1J shows sympathetic nerve fibers (TH, green), and MSCs (platelet-derived growth factor receptor alpha, PDGFRA). Scale bar: 20 μm. FIG. 1K shows GAT ILC2 in ADRB2 wild-type (Adrb2^{fl}) cells (n=6) and cells with ADRB2 deleted from glial cells (Adrb2^{ΔGfap}); n=8. FIG. 1L shows GAT ILC2 in ADRB2wild-type (Adrb2f) cells (n=10) and cells with ADRB2 deleted from MSCs (Adrb2^{ΔPdgfra}); n=8. Data are representative of 3 independent experiments. n represents biologically independent animals. Mean and error bars: standard error of the mean (s.e.m.) two-tailed unpaired Student t-test (b-g). One-way ANOVA (h). *P<0.05; **P<0.01; ***P<0.005; ****p<0.001; ns not significant.

FIGS. 2A-2O. Sympathetic cues orchestrate mesenchyme-derived glial cell line-derived neurotrophic factor (GDNF) and innate type 2 cytokines. FIGS. 2A-2C show effect of 6-OHDA treatment. FIG. 2A shows RNAseq of platelet-derived growth factor receptor alpha positive mesenchymal stem cells (PDGFRA+MSCs). Top: Mean-difference plot of vehicle versus 6-OHDA; Bottom: heatmap of downregulated genes. Vehicle n=4, 6-OHDA n=5. FIG. 2B shows Gdnf expression in total GAT RNA, n=5. FIG. 2C shows Gdnf expression in GAT cell populations, n=4. FIGS. 2D-2E show clenbuterol administration. FIG. 2D shows Gdnf expression in total GAT RNA, n=5. FIG. 2E shows Gdnf expression in GAT cell populations, n=5. FIG. 2F shows Gdnf expression in total GAT RNA in ADRB2 wild-type cells (Adrb2^{fl}, n=9) and cells with ADRB2 deleted from MSCs (Adrb2^{ΔPdgfra}). FIG. 2G shows Gdnf expression in GAT cell populations. Adrb2^{fl} n=6, Adrb2^{ΔPdgra} n=4. FIG. 2H shows GDNF median fluorescence intensity (MFI) in MSCs. n=4. FIG. 2I shows GAT staining of PDGFRA (left) and GDNF (right). Scale bar: 50 μm. FIG. 2J shows rearranged in transfection (RET) expression in ILC2 cells, T cells (T), natural killer cells (NK), B cells (B), and macrophages (Mφ). n=7. FIG. 2K shows ILC2 activity in GAT in mice expressing wild-type RET (Ret^{fl}) and Ret deleted from hematopoietic stem cells (Ret^{ΔVav1}); n=5. FIG. 2L shows ILC2 activity in GAT in Rag1 knock-out (Rag1^{-/-}) knockout mice expressing either wild-type Ret (Ret^{WT}) or mice with Ret knocked-out from IL-5 positive cells (Ret^{ΔIl5}); n=6. FIGS. 2M-2N show in vitro stimulation with GDNF. FIG. 2M shows cytokine expression in GAT ILC2. n=4. FIG. 2N shows MFI innate type 2 cytokines. UN is untreated and GDNF is stimulated with GDNF; n=6. FIG. 2O shows ILC2 activity in GAT from bone marrow (BM) chimeras. Rag1 knock-out, RET wild-type=Rag1^{-/-}, Ret^{WT} (n=5), Rag1 knock-out, RET gain of function=Rag1^{-/-}, Ret^{MEN2B}; n=4. Data are representative of 3 independent experiments. n represents biologically independent animals. Mean and error bars: s.e.m. two-tailed unpaired Student t-test (b-h, k-o). One-way ANOVA (j). *P<0.05; **P<0.01; ***P<0.005; ns not significant.

FIGS. 3A-3O. ILC2-intrinsic RET cues control adipose tissue physiology and obesity. FIGS. 3A-3M show measurements after 16 weeks on a high-fat diet (HFD) regimen in Ret wild-type (Ret^{fl}; Ret^{WT}), Ret knock-out (Ret^{ΔVav1}; Ret^{Δ}), or Ret gain-of-function (Ret^{MEN2B}) mice. FIG. 3A show weight gain; n=6. FIG. 3B show glucose tolerance test; n=6. FIG. 3C show GAT weight; n=6. FIGS. 3D-3H show ILC2 chimaeras with ILC2 RET wild-type (Ret^{WT}) or RET knock-out (Ret^{Δ}) transplants, and FIGS. 3I-3M show ILC2 Ret^{WT} or Ret^{MEN2B} transplants. FIG. 3D shows weight gain in Ret^{WT} (n=5) and Ret^{Δ} (n=6) mice. FIG. 3E shows glucose tolerance test in Ret^{WT} (n=4) and Ret^{Δ} (n=6). FIG. 3F show GAT weight in Ret^{WT} (n=4) and Ret^{Δ} (n=6). FIG. 3G shows adipocyte area in Ret^{WT} (n=4), Ret^{Δ} (n=6), Background: white 200 μm² range intervals; grey 1000 μm² range intervals. FIG. 3H shows GAT. Scale bar: 100 μm. FIG. 3I shows GAT weight. n=5. FIG. 3J shows glucose tolerance test. n=5. FIG. 3K shows GAT weight. n=5. FIG. 3L shows adipocyte area. Background: white 300 μm² range intervals; grey 1000 μm². n=5. FIG. 3M shows GAT. Scale bar: 100 μm. FIG. 3N shows uncoupling protein 1 expression (Ucp1), cytochrome c oxidase subunit 8B (Cox8b), and cell death inducing DFFA like effector A (Cidea) expression in GAT. Rag1^{-/-}Ret^{MEN2B} BM chimaeras. Rag1^{-/-}, Ret^{WT} n=4, Rag1^{-/-}, Ret^{MEN2B} n=5. Data are representative of 3 independent experiments. n represents biologically independent animals. Mean and error bars: s.e.m. Repeated measures ANOVA (FIGS. 3A, 3B, 3D, 3E, 3I, and 3J) with tests for interaction (Int), time and genotype (Gen) reported (FIGS. 3A, 3D, and 3I). Two-tailed unpaired Student t-test (FIGS. 3C, 3F, 3K). Mann-Whitney test (FIGS. 3N and 3O). *P<0.05; **P<0.01; ***P<0.005; ****p<0.001; ns is not significant.

FIGS. 4A-4N. An aorticorenal-adipose circuit that connects to the brain and regulates ILC2. FIGS. 4A-4F show viral tracing (VT, right panel) and tyrosine hydroxylase (TH, left panel). Scale bar: 50 μm. FIG. 4A shows GAT. FIG. 4B shows GAT sympathetic fibers. FIG. 4C shows genitofemoral (GF) nerve fibers (arrows). FIG. 4D shows TH positive fibers of the genitofemoral nerve. FIG. 4E shows aorticorenal ganglion (ARG, circled). FIG. 4F shows TH positive neuronal cell bodies in the aorticorenal ganglion. FIG. 4G shows Left: a brain atlas scheme of coronal section and Right: PRV-RFP viral tracing from the GAT corresponding to the highlighted area on the left. PVH is paraventricular nucleus of the hypothalamus FIG. 4H shows Left: a brain atlas scheme of coronal section and Right: a PRV-RFP viral tracing from the aorticorenal ganglion (ARG) corresponding to the highlighted area on the left. Scale bar 200 μm (FIGS. 4G, 4H). FIG. 4I shows surgical genitofemoral nerve (GF) ablation (GFx) scheme (left) and GAT Gdnf expression (right). n=4. FIG. 4J shows GAT ILC2 activity in control (Sham) and genitofemoral nerve ablation (GFx). n=5. FIG. 4K shows Left: chemogenetic inhibition scheme with adeno-associated virus 4D (AAV 4D) and Right: GAT Gdnf expression. n=5. FIG. 4L shows GAT ILC2 activity in mice with Gdnf expression inhibited by AAV 4D. n=5. FIG. 4M shows Left: chemogenetic activation scheme with adeno-associated virus 3D (AAV 3D) and Right: GAT Gdnf expression (right). n=4. FIG. 4N, GAT ILC2 activity in mice with Gdnf expression activated by AAV 3D. n=4. Data are representative of 3 independent experiments. n represents biologically independent animals. Mean and error bars: s.e.m. two-tailed unpaired Student t-test. *P<0.05; **P<0.01; ***P<0.005; ns not significant.

FIGS. 5A-5D. Sympathetic nervous system in the GAT and ILC2 function. FIG. 5A shows GAT with stained sympathetic nerve fibers (TH) and endothelial cells (CD31). Scale bar: 300 μm. FIG. 5B shows GAT ILC2-derived Met-Enk production after 6-OHDA administration. n=5. FIG. 5C shows CD4 T cells and TH positive CD4 T cells after 6-OHDA administration. n−4. FIG. 5D shows GAT ILC2-derived Met-Enk after clenbuterol administration. n=5. Data are representative of 3 independent experiments. n represents biologically independent animals. Mean and error bars: s.e.m. two-tailed unpaired Student t-test. *P<0.05; ***P<0.005.

FIGS. 6A-6E. Sympathetic regulation of GAT mesenchymal stem cells (MSC). FIG. 6A shows a heatmap of upregulated and downregulated genes in MSC upon 6-OHDA administration. Vehicle n=4, 6-OHDA n=5. FIG. 6B shows total GAT Il33 expression after 6-OHDA treatment. n=5. FIG. 6C shows GAT Il33 expression after Clenbuterol administration. n=5. FIG. 6D shows MSC-derived Il33 expression in PDGFRA+MSCs after 6-OHDA and clenbuterol administration. n=6. FIG. 6E shows MSC-derived Il25 expression in PDGFRA+MSCs after 6-OHDA and Clenbuterol administration. n=6. Data are representative of 3 independent experiments. n represents biologically independent animals. Mean and error bars: s.e.m. two-tailed unpaired Student t-test. ns is not significant.

FIGS. 7A-7L. ILC2-autonomous RET signals control type 2 innate cytokines in the GAT. FIGS. 7A-7C show GAT ILC2 function in GDNF Family Receptor Alpha (GFRa) mice. FIG. 7A shows GAT ILC2 function in GFR1 Alpha positive (Gfra1^{+/+}) and knock-out (Gfra1^{-/-}) foetal liver chimaeras. n=5. FIG. 7B shows GFR2 Alpha positive (Gfra2^{+/+}, n=10) and knock-out (Gfra2^{-/-} n=5) mice. FIG. 7C shows GFR3 Alpha positive (Gfra3^{+/+} n=8); and knock-out (Gfra3^{-/-} n=8) mice. FIG. 7D shows scheme to produce mixed bone marrow (BM) chimaeras of Rag1 knock-out, interleukin 2 receptor gamma knock-out (Rag1^{-/-}, Il2rg^{-/-}) and Ret^{ΔVav1} mice. FIG. 7E shows GAT ILC2 activity from Ret floxed (Ret^{fl}), Rag1^{-/-}, Il2rg^{-/-} mixed BM chimeras and from Ret knock-out (Ret^{ΔVav1}), Rag1^{-/-}, Il2rg^{-/-} mixed BM chimaeras. Ret^{fl} n=6; Ret^{ΔVav1} n=7. FIG. 7F shows ILC2 activity in mice with Rag1 knock-out, Ret wild-type (Rag1^{-/-}, Ret^{WT} n=6) and Rag1 knock-out, Ret knock-out in interleukin 5 cells (Rag1^{-/-}·Ret^{ΔIl5} n=6). FIG. 7G shows ILC2 activity in mice. Ret^{WT} n=10 and Ret^{ΔIl5} (n=8). FIG. 7H shows scheme to produce mixed bone marrow (BM) chimeras of Rag1 knock-out, interleukin 2 receptor gamma knock-out (Rag1^{-/-}, Il2rg^{-/-}) and Ret^{ΔIl5} mice. FIG. 7I shows ILC2 activity in Ret wild-type (Ret^{WT}, n=4) mixed BM chimeras and Ret knock-out IL5 cells (Ret^{ΔIl5} n=4). FIG. 7J shows GAT ILC2 in Rag1^{-/-}, Ret wild-type (Ret^{WT}) and Ret gain-of-function (Ret^{MEN2B}) mixed bone marrow (BM) chimaeras. Rag1^{-/-}, Ret^{WT} n=5, Rag1^{-/-}·Ret^{MEN2B} n=6. FIG. 7K shows mixed bone marrow (BM) chimaeras scheme to produce Ret^{MEN2B} mixed BM chimaeras. FIG. 7L shows ILC2 activity in mixed BM chimaeras in Rag1 knock-out and Ret wild-type mice (Rag1^{-/-}, Ret^{WT}; n=6) and (Rag1^{-/-}·Ret^{MEN2B}; n=7). Data are representative of 3 independent experiments. n represents biologically independent animals. Mean and error bars: s.e.m. two-tailed unpaired Student t-test. *P<0.05; **P<0.01; ***P<0.005; ns not significant.

FIGS. 8A-8E. ILC2-intrinsic RET signalling is sufficient to control adipocyte physiology and obesity. FIG. 8A shows GAT ILC2 function after 6-OHDA administration. Ret^{WT} n=8 and Ret^{ΔIl5} n=7. FIGS. 8B, 8C show measurements after 16 weeks on a high-fat diet (HFD) regimen in Ret wild-type (Rag1^{-/-}·Ret^{WT}; Ret^{WT}) and Ret knock-out (Rag1^{-/-}·Ret^{ΔIl5}; Ret^{ΔIl5}). FIG. 8B shows weight gain during 16 weeks of HFD regimen. Rag1^{-/-}·Ret^{WT} n=4, Rag1^{-/-}·Ret^{ΔIl5} n=5. FIG. 8C shows weight gain during 16 weeks of HFD regimen. Ret^{WT} n=5, Ret^{ΔIl5} n=5. FIG. 8D shows total GAT RNA expression of Ucp1, Cox8b and Cidea. n=5.

FIG. 8E shows GAT RNA expression of Ucp1 in GAT explant co-culture with Ret^{WT} and Ret knock-out (Ret^{Δ}) cells stimulated with glial-derived neurotrophic factor (GDNF). Mean and error bars: s.e.m. two-tailed unpaired Student t-test (FIG. 8A); repeated measures ANOVA (FIGS. 8B, 8C); Mann Whitney test (FIGS. 8D, 8E). *P<0.05; **P<0.01; ns not significant; unstim is unstimulated with GDNF.

FIGS. 9A-9G. An aorticorenal-adipose circuit connects to the brain. FIG. 9A shows dorsal root ganglion (DRG) T13 viral tracing (VT) and tyrosine hydroxylase (TH) staining. Scale bar: 100 μm. FIG. 9B shows Left: Brain atlas scheme of coronal section. Right: Polysynaptic tracing from the GAT corresponding to the highlighted area on the left. FIG. 9C shows Left: Brain atlas scheme of coronal section. Right: Polysynaptic tracing from the Aorticorenal ganglion (ARG) corresponding to the highlighted area on the left. FIGS. 9B-9C, Central amygdala (CA), Zona Incerta (ZI), Periaquedutal Gray (PAG) and Subcoeruleus Nucleus (SubCD). FIG. 9D shows electrolytic lesion (electroablation) of the PVH. Scale bar 500 μm. FIG. 9E shows GAT ILC2 in control (Sham) and PVH ablated (Abl) mice. Sham n=5; PVH ablated n=6. FIG. 9F shows GAT Il33 expression in AAV (4D) compared to contralateral control after CNO administration. n=5. FIG. 9G shows GAT Il33 expression in AAV (3D) compared to contralateral control after CNO administration. n=4. Data are representative of 3 independent experiments. n represents biologically independent animals. Mean and error bars: s.e.m. two-tailed unpaired Student t-test. *P<0.05; **P<0.01.

FIG. 10. A sympathetic aorticorenal-adipose circuit connects to the brain and regulates ILC2. GAT neuro-mesenchyme units translate sympathetic cues into neurotrophic factor expression. In turn, neurotrophic factors control adipose ILC2 function via the neuroregulatory receptor RET, shaping the host metabolism, energy expenditure and obesity. PVH-Paraventricular nucleus of the hypothalamus; SNS-Sympathetic nervous system; ARG-Aorticorenal ganglion.

### DETAILED DESCRIPTION

Obesity results from an excessive accumulation of lipid depots, and these fat reservoirs can be used as a high-energy source during periods of dietary deprivation. Sympathetic neuronal cues drive lipolysis during dietary deprivation,⁵⁻⁷ and ILC2s contribute to visceral adipose tissue metabolism via production type 2 innate cytokines and Met-enkephalin (Met-Enk)⁸⁻¹³. This raises the hypothesis that the nervous system and ILC2 cooperate by previously-unappreciated mechanisms to sense metabolic stress and to drive adipose physiology via higher-order brain interactions.

A newly-discovered neuro-mesenchyme unit is described herein that controls ILC2s, adipose tissue physiology, metabolism, and obesity via a brain-adipose circuit. Sympathetic neurons in this brain-adipose circuit act on neighboring adipose mesenchymal cells via the beta-2 adrenergic receptor (ADRB2) to control the expression of glial-derived neurotrophic factor (GDNF) and the activity of gonadal fat ILC2. Accordingly, ILC2-autonomous manipulation of the GDNF receptor machinery leads to altered ILC2 function, energy expenditure, insulin resistance, and propensity to obesity. Retrograde tracing, chemical, surgical, and chemogenetic manipulations identified an unappreciated sympathetic aorticorenal circuit that modulates gonadal fat ILC2 and connects to higher-order brain areas, including the paraventricular nucleus of the hypothalamus (PVH).

Therefore, the methods provided herein manipulate a newly-discovered neuro-mesenchymal unit that translates long-range neuronal circuitry cues into adipose-resident ILC2 function, shaping host metabolism and obesity.

### Methods of Use

In some aspects, the methods provided herein increase the activity, proliferation, or activity and proliferation of Group 2 innate lymphoid cells (ILC2s) by contacting ILC2s with a rearranged during transfection (RET) agonist, contacting mesenchymal stromal cells (MSCs) with a beta-2-adrenergic receptor (ADRB2) agonist, or contacting ILC2s with a RET agonist and contacting MSCs with an ADRB2 agonist.

In some embodiments, the present disclosure provides methods for increasing the activity or proliferation of ILC2s. ILC2s are a subset of innate lymphocytes that are important in maintaining tissue homeostasis and regulating lymphoid tissue development, tissue repair, and fat metabolism. ILC2s are abundant at mucosal barriers in adipose tissue, lung, small intestine, large intestine, mesenteric lymphoid nodes, bone marrow, spleen, liver, and kidney, where they act as key initiators of type 2 inflammation and tissue repair. They are activated by cytokines, including interleukin-25 (IL-25), interleukin-33 (IL-33), and thymic stromal lymphopoietin.

Any activity of ILC2s may be increased by methods provided herein. Non-limiting examples of ILC2 activity that may be increased include: adipose tissue metabolism, tissue homeostasis, defense against parasites, tissue repair, inflammation, and immunopathology associated with type-2 immunity. ILC2 activity may be measured by any method known in the art including, but not limited to: quantitative PCR measurement and fluorescence quantification of proteins produced by ILC2 cells (e.g., cytokines). In some embodiments, methods provided herein increase the activity of ILC2s in adipose tissue metabolism. ILC2 activity (e.g., adipose tissue metabolism) may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 activity is increased by at least 5%, 10%, 25%, 50%, 75%, 100%, 150%, 200%, 250%, 300%, 350%, 400%, 450%, 500%, 550%, or more relative to a control. A control may be an ILC2 that is not contacted with a RET agonist or the same ILC2 before it is contacted with a RET agonist.

In some embodiments, ILC2 proliferation is increased after contact with a RET agonist. ILC2 proliferation refers to the growth and replication of ILC2s. ILC2 proliferation may be measured by any method known in the art including, but not limited to: immunohistochemistry of ILC2 surface proteins and quantitative PCR of ILC2-specific proteins (e.g., RET receptor, neuropeptide receptor Nmur1, interleukin-33 receptor ST2, IL-17A/IL-17B receptor). ILC2 proliferation may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 proliferation is increased by at least 5%, 10%, 25%, 50%, 75%, 100%, 150%, 200%, 250%, 300%, 350%, 400%, 450%, 500%, 550%, or more relative to a control. A control may be an ILC2 that is not contacted with a RET agonist or the same ILC2 before it is contacted with a RET agonist.

### RET Agonists

In some embodiments, methods provided herein include contacting an ILC2 with a Rearranged during Transfection (RET) agonist. RET is a receptor tyrosine kinase for members of the glial cell line-derived neurotrophic factor (GDNF) family of extracellular signaling molecules. RET loss of function mutations are associated with the development of Hirschsprung's disease, and RET gain of function mutations are associated with the development of various types of human cancer, including medullary thyroid carcinoma, multiple endocrine neoplasias type 2A and 2B, pheochromocytoma, and parathyroid hyperplasia.

RET is also known as cadherin family member 12, cadherin-related family member 16, CDHF12, CDHR16, HSCR1, hydroxyaryl-protein kinase, MEN2A, MEN2B, MTC1, PTC, ret proto-oncogene, RET-ELE1, RET/PTC, RET51, and RET-HUMAN. The amino acid sequence of RET can be found at e.g., UniProtKB P07949; it has two isoforms, P07949-1 (isoform 1) and P07949-2 (isoform 2). The nucleotide sequence can be found at e.g., AK291807 (mRNA/cDNA sequence).

A RET agonist is a compound that binds and increases the activity of a RET protein relative to a control. A control may be a measurement taken from an ILC2 before it is contacted with a RET agonist, a measurement taken from an ILC2 in the same sample (e.g., in vitro or in vivo) that is not contacted with a RET agonist, or a sample that is not contacted with a RET agonist. A RET agonist may increase the activity of a RET protein by at least 10%, 25%, 50%, 100%, 200%, 300%, 400%, 500%, 600%, 700%, 800%, 900%, 1000%, or more compared to a control.

A RET agonist herein may be any RET agonist known in the art. Non-limiting examples of RET agonists include (1) a combination of a soluble glial-derived neurotrophic factor (GDNF) Family binding Receptor alpha (GFRα) and a GFRα ligand (GFL) or an analog or mimetic thereof; or (2) an antibody or an antigen-binding fragment thereof that specifically binds to RET and increases RET tyrosine kinase activity.

A RET agonist may be entirely specific to RET, may preferentially agonize RET as compared to other tyrosine kinases, or may agonize both RET and other tyrosine kinases. These agonists may be useful even if RET is agonized less than other tyrosine kinases, but it is preferable that the agonists used in methods described herein agonize RET to a great extent than other tyrosine kinases. As used herein, agonizing RET preferentially (as compared to other tyrosine kinases) means that the agonist agonizes RET at least 10%, 25%, 50%, 100%, 200%, 300%, 400%, 500%, 600%, 700%, 800%, 900%, 1000%, or more than other tyrosine kinases.

A combination of a soluble GFRα and a GFL or an analog or mimetic thereof may include any soluble GFRα or GFL (or analog or mimetic thereof) known in the art. Non-limiting examples of soluble GFRα and GFL include: soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

Soluble GFRα molecules and GFLs include any GFRαs and GFLs known in the art and described herein, e.g., GFRα1, GFRα2, GFRα3, and GFRα4; and their respective ligands GDNF, neurturin (NRTN), artemin (ARTN), and persephin (PSPN). Analogs, mimetics, derivatives, and conjugates of GFRαs and GFLs include GFRα and GFL analogs having variation in amino acid sequences relative to natural (e.g., endogenous) GFRα and GFL sequences but which retain the function of activating RET.

In some embodiments, a soluble GFRα molecule is GFRα1. GFRα1 is also known as GDNF receptor, GDNFR, GDNFRA, GFR-ALPHA-1, RETIL, RETL1, TRNR1, and GDNF family receptor alpha 1. The amino acid sequence of GFRα1 can be found at e.g., UniProtKB P56159; it has two isoforms, P56159-1 (isoform 1) and P56159-2 (isoform 2). The nucleotide sequence can be found at e.g., AF042080.1 (mRNA/cDNA sequence).

In some embodiments, a soluble GFRα molecule is GFRα2. GFRα2 is also known as neurturin receptor, GFRA2, GDNFRB, NRTNR-ALPHA, NTNRA, RETL2, TRNR2, and GDNF family receptor alpha 2. The amino acid sequence of GFRα2 can be found at e.g., UniProtKB 000451; it has three isoforms, 000451-1 (isoform 1), 000451-2 (isoform 2), and 000451-3 (isoform 3). The nucleotide sequence can be found at, e.g., AY326396 (mRNA/cDNA sequence).

In some embodiments, a soluble GFRα molecule is GFRα3. GFRα3 is also known as artemin receptor, GFRA3, GDNFR3, and GDNF family receptor alpha. The amino acid sequence of GFRα3 can be found at e.g., UniProtKB 060609; it has two isoforms, 060609-1 (isoform 1) and 060609-2 (isoform 2). The nucleotide sequence can be found at e.g., AK297693 (mRNA/cDNA sequence).

In some embodiments, a soluble GFRα molecule is GFRα4. GFRα4 is also known as persephin receptor and GFRA4. The amino acid sequence of GFRα4 can be found at, e.g., UniProtKB Q9GZZ7; it has three isoforms, Q9GZZ7-1 (isoform GFRalpha4b), Q9GZZ7-2 (isoform GFRalpha4a), and Q9GZZ703 (isoform GFRalpha4c). The nucleotide sequence can be found at e.g., AF253318.

In some embodiments, a GFL is glial cell-derived neurotrophic factor (GDNF). GDNF is also known as ATF1, ATF2, HFB1-HSCR3, and glial cell derived neurotrophic factor. The amino acid sequence can be found at, e.g., UniProtKB P39905; it has three isoforms, P39905-1 (isoform 1), P39905-2 (isoform 2), P39905-3 (isoform 3), P39905-4 (isoform 4), and P39905-5 (isoform 5). The nucleotide sequence can be found at e.g., CR541923 (mRNA/cDNA sequence).

In some embodiments, a GFL is neurturin (NRTN). The amino acid sequence can be found at, e.g., UniProtKB Q99748. The nucleotide sequence can be found at e.g., BC137399 (mRNA/cDNA sequence).

In some embodiments, a GFL is artemin (ARTN), which is also known as enovin, neublastin, EVN, and NBN. The amino acid sequence can be found at, e.g., UniProtKB Q5T4W7; it has three isoforms, Q5T4W7-1 (isoform 1), Q5T4W7-2 (isoform 2), and Q5T4W7-3 (isoform 3). The nucleotide sequence can be found at, e.g., AF109401 (mRNA/cDNA sequence).

In some embodiments, a GFL is persephin (PSPN). The amino acid sequence can be found at, e.g., UniProtKB 060542. The nucleotide sequence can be found at, e.g., AF040962 (mRNA/cDNA sequence).

Examples of analogs, derivatives, and conjugates of GFLs include: the variants of GDNF which retain an GDNF receptor agonist function described in U.S. Pat. No. 9,133,441; the variants of GDNF described in U.S. Pat. No. 9,243,046; the GFL variants (e.g. AN-GDNF) that efficiently activate RET but lack heparin-binding sites and do not interact with HSPGs in extracellular matrix described in U.S. Pat. No. 8,034,572; the neurturin molecules that have reduced heparin, heparan sulfate and heparan sulfated proteoglycan binding ability but retain the ability to induce phosphorylation of the RET protein described in U.S. Pat. Nos. 8,445,432, 9,127,083 and 9,469,679; the GDNF derived peptides described in U.S. Pat. No. 8,138,148; the neublastin molecules and dimerized proteins described in U.S. Pat. Nos. 7,276,580, 7,598,059 and 7,655,463; and the chimeric GDNF family ligands which activate GFRα/RET described in U.S. Pat. No. 6,866,851.

Other examples of analogs, derivatives, and conjugates of GFLs include: the GDNF analogs described in WO 2012/151476, EP 2440581, and other patent publications referenced therein, isoforms, precursors, fragments and splice variants of GDNF, such as those described in WO 2009/053536, US 2009/0069230, WO 2008/069876, WO 2007/019860, and US 2006/0258576.

Still other agonists of RET include the GDNF family ligands (GFL) and mimetics or RET signaling pathway activators and direct RET activators described in U.S. Pat. No. 8,901,129.

Another agonist of RET is a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035). As shown by Tokugawa et al. (Neurochem Int. 2003 January; 42(1):81-6), XIB4035, like GDNF, induced RET autophosphorylation. A chemical structure of XIB4035 is shown below:

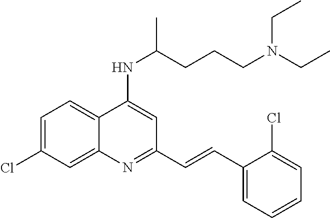 [SMILES: CCN(CC)CCCC(C)Nc1cc(/C=C/c2ccccc2Cl)nc2cc(Cl)ccc12]

Another agonist of RET is a soluble GFRα and a BT compound. BT compounds are described in WO 2011/070177.

Another agonist of RET is a soluble GFRα and an antibody that specifically binds to and dimerizes the GFRα. Antibodies that specifically bind to a GFRα and dimerize the GFRα can be obtained by screening for this activity among a set of GFRα-binding antibodies.

Additional agonists of RET are antibodies that specifically bind to RET and increase RET tyrosine kinase activity or an antigen-binding fragment of such antibodies. RET-binding antibodies are known in the art, such as those described in U.S. Pat. No. 6,861,509, and various commercially-available antibodies. Antibodies that specifically bind to RET and increase RET tyrosine kinase activity can be obtained by screening for this activity among a set of RET-binding antibodies.

Additional agonists of RET include multikinase inhibitors, including but not limited to cabozanitib, levatinib, sunitinib, and alectinib. Still further agonists of RET include the selective RET inhibitors selpercatinib (LOXO-292), pralsetinib (BLU-667), BOS172738 (Boston Pharmaceuticals), HM06 (Helsinn), TPX-0046 (Turning Point Therapeutics), LOX-18228 (Eli Lilly), osimertinib, RXDX-105 (Hoffmann-La Roche), regorafenib, RPI1, and GSK3352589.

An ILC2 cell may be contacted by more than one RET agonist. In some embodiments, an ILC2 cell is contacted by one-ten, two-nine, three-eight, four-seven, or five-six RET agonists. In some embodiments, an ILC2 cell is contacted with one, two, three, four, five, six, seven, eight, nine, ten, or more RET agonists. In embodiments wherein an ILC2 is contacted with multiple (e.g., two or more) RET agonists, the ILC2 may be contacted with the multiple RET agonists simultaneously or sequentially.

### Mesenchymal Stromal Cells

In some aspects, methods of the present disclosure provided herein include contacting mesenchymal stromal cells (MSCs) with a beta-2-adrenergic receptor (ADRB2) agonist. MSCs are spindle-shaped, fibroblast-like cells isolated from bone marrow, adipose, and other tissue sources, with multipotent differentiation capacity in vitro. MSCs can differentiate into chondrocytes, osteoblasts, adipocytes, myoblasts, and other cell types. MSCs express adrenergic receptors (α1A, α1B, α2A, α2B, β1, β2, and β3), CD90, CD105, and CD73 on the surface but do not express CD45, CD34, CD14, CD11b, CD79a, CD19, and HLA-DR.

In some embodiments, methods of the present disclosure provided herein include increasing activity of MSCs, increasing proliferation of MSCs, or increasing activity and proliferation of MSCs. Any activity of MSCs may be increased by methods provided herein.

Non-limiting examples of MSC activity that may be increased include: differentiation into other cell types (e.g., adipocytes, chrondrocytes, osteoblasts, adipocytes, myoblasts, and other cell types), extracellular collagen production, and alkaline phosphatase activity. MSC activity may be measured by any method known in the art including, but not limited to: quantitative PCR measurement of proteins produced by MSCs (e.g., adipogenic proteins including, but not limited to: AP-1, KLF4, KLF6, C/EBPα, C/EBPβ, C/EBPδ, PPARγ, STAT5A, SREBP-1), cellular morphology changes during differentiation (e.g., spindle-shaped MSC changing into round adipocytes), and cellular cytoskeleton restructuring during differentiation. In some embodiments, methods provided herein increase the activity of MSCs in adipocyte differentiation. MSC activity may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, MSC activity is increased by at least 5%, 10%, 25%, 50%, 75%, 100%, 150%, 200%, 250%, 300%, 350%, 400%, 450%, 500%, 550%, or more relative to a control. A control may be an MSC that is not contacted with an ADRB2 agonist or the same MSC before it is contacted with an ADRB2 agonist.

In some embodiments, MSC proliferation is increased after contact with an ADRB2 agonist. MSC proliferation may be measured by any method known in the art including, but not limited to: immunohistochemistry of MSC surface proteins (e.g., adrenergic receptors (α1A, α1B, α2A, α2B, β1, β2, and β3), CD90, CD105, and CD73), quantitative PCR of MSC-specific proteins (e.g., adrenergic receptors (α1A, α1B, α2A, α2B, β1, β2, and β3), CD90, CD105, and CD73), and quantification of cell proliferation markers (e.g., Ki67, PCNA). MSC proliferation may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, MSC proliferation is increased by at least 5%, 10%, 25%, 50%, 75%, 100%, 150%, 200%, 250%, 300%, 350%, 400%, 450%, 500%, 550%, or more relative to a control. A control may be a MSC that is not contacted with a ADRB2 agonist or the same MSC before it is contacted with an ADRB2 agonist.

### Beta-2-Adrenergic Receptor (ADRB2) Agonist

In some aspects, methods provided herein include contacting an MSC with a beta-2-adrenergic receptor (ADRB2) agonist. ADRB2 is a cell membrane-spanning beta-adrenergic receptor that binds epinephrine, which mediates downstream physiologic responses such as smooth muscle relaxation and bronchodilation. ADRB2 functions in the human muscular system, circulatory system, optic system, digestive system, immune system, and respiratory system. ADRB2 is believed to be associated with risk of Parkinson's disease and different polymorphic forms, point mutations, and/or downregulation of this gene are associated with nocturnal asthma, obesity, type 2 diabetes, and cardiovascular disease.

ADRB2 is also known as adrenoreceptor beta 2, B2AR, beta-2 adrenoreceptor, beta-2 adrenoceptor, and catecholamine receptor. The amino acid sequence of ADRB2 can be found at e.g., UniProtKB P07550-1. The nucleotide sequence can be found at e.g., X04827 (mRNA/cDNA sequence).

An ADRB2 agonist is a compound that binds and increases the activity of an ADRB2 protein relative to a control. A control may be a measurement taken from an MSC before it is contacted with an ADRB2 agonist, a measurement taken from an MSC in the same sample (e.g., in vitro or in vivo) that is not contacted with an ADRB2 agonist, or a sample that is not contacted with an ADRB2 agonist. An ADRB2 agonist may increase the activity of a MSC protein by at least 10%, 25%, 50%, 100%, 200%, 300%, 400%, 500%, 600%, 700%, 800%, 900%, 1000%, or more compared to a control.

In some embodiments, an ADRB2 agonist is a short-acting beta-agonist (SABA). A SABA has effects that last 4 hours-6 hours, depending on the agonist. A SABA is a first-line medication for acute treatment and are commonly used in conjunction with other compounds (e.g., long-acting beta-agonists (LABAs), corticosteroids). Non-limiting examples of SABAs include: bitolterol (Tornalate), fenoterol (Berotec), isopreoterenol, levalbuterol, metaproterenol, pirbuterol, procaterol, ritodrine (Yutopar), albuterol (Ventolin/Proventil), and terbutaline (Bricanyl).

In some embodiments, an ADRB2 agonist is a long-acting beta-agonist (LABA). A LABA is most commonly used in combination with a steroid and has effects that last 12 hours-24 hours, depending on the agonist. Non-limiting examples of LABA include: arfomoterol (Brovana), bambuterol (Bambec/Oxeol), clenbuterol (Dilaterol/Spiropent), formoterol (Foradil/Oxis/Perforomist), and salmeterol (Serevent).

In some embodiments, an ADRB2 agonist is an ultra-long-acting beta-agonist (ULABA). An ULABA has effects that last longer than 24 hours, and the duration of their effects will depend on the agonist. Non-limiting examples of ULABA include: abediterol, carmoterol, indacaterol (Arcapta Neohaler), olodaterol (Striverdi Respimat), and vilanterol.

In some embodiments, an ADRB2 agonist has an unknown duration of action. Non-limiting examples of ADRB2 agonists of unknown duration of action include: isoxsuprine, mabuterol, and zilpaterol.

A MSC may be contacted by more than one ADRB2 agonist. In some embodiments, a MSC is contacted by one-ten, two-nine, three-eight, four-seven, or five-six ADRB2 agonists. In some embodiments, a MSC is contacted with one, two, three, four, five, six, seven, eight, nine, ten, or more ADRB2 agonists. In embodiments wherein a MSC is contacted with multiple (e.g., two or more) ADRB2 agonists, the MSC may be contacted with the multiple ADRB2 agonists simultaneously or sequentially.

### Cytokine Production

Provided herein, in some aspects, are methods for increasing cytokine production. Cytokine production can be from an ILC2, an MSC, or any other cell that produces cytokines (e.g., T cells, B cells, macrophages, mast cells, endothelial cells, fibroblasts). In some embodiments, a RET agonist provided herein increases cytokine production from an ILC2 cell. In some embodiments, an ADRB2 agonist provided herein increases cytokine production from an MSC. In some embodiments, a RET agonist provided herein increases cytokine production from an ILC2 cell and an ADRB2 agonist provided herein increases cytokine production from an MSC.

Cytokines are small (˜5-20 kilodaltons) peptides used in cellular signaling by binding to receptors on a target cell. Cytokines are important in host immune responses to infection, inflammation, trauma, sepsis, cancer, and reproduction. A cytokine may be a type 1 cytokine that enhances cellular responses (e.g., TNFα, IFNγ) and a type 2 cytokine that enhances antibody responses (e.g., TGF-β, IL-4, IL-10, IL-13). Cytokine production may be measured by any method known in the art including, but not limited to: immunofluorescence staining of cytokines, enzyme-linked immunosorbent assays (ELISAs), enzyme-linked immunosorbent spot (ELIspot) assays, antibody array assays, and bead-based assays.

Non-limiting examples of cytokines that may be increased by methods disclosed herein include: interleukin-5 (IL-5), interleukin-13 (IL-13), Met-enkephalin (Met-Enk), amphiregulin, interleukin-4 (IL-4), interleukin-9 (IL-9), eotaxin, interferon gamma-induced protein 10 (IP-10), vascular endothelial growth factor (VEGF), TIMP metallopeptidase inhibitor 1 (TIMP1), adipocyte lipid binding protein (ALBP), and fatty acid translocase (FAT/CD36).

In some embodiments, methods provided herein increase cytokine production. Cytokine production may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, cytokine production is increased by at least 5%, 10%, 25%, 50%, 75%, 100%, 150%, 200%, 250%, 300%, 350%, 400%, 450%, 500%, 550%, or more relative to a control. A control may be an ILC2 that is not contacted with a RET agonist, a MSC that is not contacted with an ADRB2 agonist, or an ILC2 that is not contacted with a RET agonist and a MSC that is not contacted with an ADRB2 agonist or the same ICL2 before it is contacted with a RET agonist and/or a MSC that is not contacted with an ADRB2 agonist.

### Methods of Treatment

### Obesity and Adipose Homeostasis

Also provided herein, in some aspects, are methods of decreasing susceptibility to obesity, increasing adipose homeostasis, or decreasing susceptibility to obesity and increasing adipose homeostasis in a subject in need thereof. Methods of decreasing susceptibility to obesity and/or increasing adipose homeostasis may include contacting an ILC2 (e.g., in a subject) with any RET agonist provided herein, contacting a MSC with any ADRB2 agonist provided herein, or a combination thereof compared to a control. A control may be a subject with ILC2s that are not contacted with a RET agonist, a subject with MSCs that are contacted with an ADRB2 agonist, or the same subject with an ICL2 before it is contacted with a RET agonist and/or an MSC before it is contacted with an ADRB2 agonist.

A subject in need thereof may be any subject that has obesity or increased susceptibility to obesity. Obesity is a disorder involving excessive body fat that increases the risk of health problems, including, but not limited to: cardiovascular disease, diabetes, high blood pressure, and high cholesterol. Obesity occurs when a person's body mass index (BMI) is 30 or over. BMI is calculated by dividing a subject's weight in kilograms by the square of their height in meters (United States Center for Disease Control) for adults 18 years or older, while factoring in gender and age for children younger than 18 years. Obesity is generally self-diagnosed by calculating BMI, and subjects with obesity may have any symptom(s) including, but not limited to: pain in the back or joints, binge eating, fatigue, sleep apnea, and excessive body fat. Conventional treatment for obesity includes, but is not limited to, physical exercise, low-fat diet (less than 30% daily calorie consumption), and behavior therapy.

In some embodiments, methods provided herein decrease susceptibility in a subject to obesity. Susceptibility to obesity may be determined by any metric known in the art, including evaluating behavior, environment, and genetic factors. Behavioral risk factors that increase susceptibility to obesity include consuming foods high in saturated fats and trans fats (>30% of daily calorie consumption) and being inactive (no physical activity beyond that of daily living). Environmental risk factors that increase susceptibility to obesity include availability of foods low in saturated fat and trans fats (<30% of daily calorie consumption), inability to be physically active, and prenatal and postnatal (within 1 year of life) maternal influences. Genetic risk factors that increase susceptibility to obesity include mutations in genes that encode for the hormone leptin, the leptin receptor, pro-opiomelanocortin, and the melanocortin-4 receptor; fat mass; and mutations in genes encoding obesity-associated protein (FTO), transmembrane protein 18 (TMEM18), glucosamine 6-phosphate deaminase 2 (GNPDA2), brain-derived neurotrophic factor (BDNF), neuronal growth regulator 1 (NEGRI), SH2B adaptor protein 1 (SH2B1), ETS variant transcription factor 5 (ETV5), mitochondrial carrier 2 (MTCH2), potassium channel tetramerization domain containing 15 (KCTD15), Fas apoptotic inhibitory molecule 2 (FAIM2), SEC homology B (SEC16B), TNNI3 interacting kinase (TNNI3K), leucine-rich repeat protein, neuronal 6C (LRRN6C), 3-hydroxy-3-methylglutaryl-coA reductase (HMGCR), protein kinase D1 (PRKD1), gastric inhibitory polypeptide receptor (RBJ/GIPR), solute carrier family 39 member 8 (SLC39A8), transmembrane protein 160 (TMEM160), Fanconi Anemia complementation group L (FANCL), cell adhesion molecule 2 (CADM2), LDL receptor protein 1B (LRP1B), polypyrimidine tract binding protein 2 (PTBP2), mitochondrial translational initiation factor 3 (MTIF3), zinc finger 608 (ZNF608), and tubby protein homolog (TUB).

In some embodiments, methods provided herein decrease susceptibility to obesity. Susceptibility to obesity may be measured by any method known in the art including, but not limited to, body fat (e.g., adipose tissue) loss, decreased saturated fat and trans fat consumption, and increased physical activity. Susceptibility to obesity may be decreased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, susceptibility to obesity is decreased by at least 5%, 10%, 25%, 50%, 75%, 100%, 150%, 200%, 250%, 300%, 350%, 400%, 450%, 500%, 550%, or more relative to a control. A control may be a subject that is not treated with methods provided herein.

In some embodiments, methods provided herein increase adipose homeostasis in a subject in need thereof. As used herein, adipose tissue homeostasis refers to balance between storing excess calories as triglycerides in white adipocytes and utilizing stored excess calories from white adipocytes during calorie withdrawal. An imbalance in adipose homeostasis occurs when more excess calories are stored as triglycerides than are necessary, when calorie withdrawal does not occur, or when more excess calories are stored as triglycerides than are necessary and calorie withdrawal does not occur. Non-limiting symptoms of an imbalance in adipose homeostasis include: decreased glucose tolerance, decreased gonadal adipose tissue mass, and increased hyperglycemia.

In some embodiments, a subject herein with decreased adipose homeostasis has decreased glucose tolerance. Decreased glucose tolerance means that a subject has glucose levels of 140 to 199 mg per deciliter (mg/dL) (7.8 to 11.0 mmol) after two hours in a 75-g glucose tolerance test. Untreated decreased glucose tolerance is also known as prediabetes and is likely to progress to type 2 diabetes without intervention.

In some embodiments, methods provided herein increase glucose tolerance. Glucose tolerance may be measured by any method known in the art including, but not limited to, a 75-gram glucose tolerance test. Glucose tolerance may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, glucose tolerance is increased by at least 5%, 10%, 25%, 50%, 75%, 100%, 150%, 200%, 250%, 300%, 350%, 400%, 450%, 500%, 550%, or more relative to a control. A control may be a subject that is not treated with methods provided herein.

In some embodiments, a subject herein with decreased adipose homeostasis has increased adipose tissue mass. Adipose tissue may be subcutaneous adipose tissue (SAT) or visceral adipose tissue (VAT). VAT surrounds inner organs in a subject and may be gonadal adipose tissue (GAT), omental adipose tissue (OAT), retroperitoneal adipose tissue (RAT), mesenteric adipose tissue (MAT), or pericardial adipose tissue (PAT). In some embodiments, increased adipose tissue mass is increased GAT mass. GAT mass is found around the testis of males (epididymal) and around the ovaries of females (periovarian). GAT expresses more PPARγ and SREBP1C and the adipogenic transcription factor CCAAT enhancer-binding protein alpha (C/EBP-alpha) genes compared to SAT.

In some embodiments, methods provided herein decrease adipose tissue (e.g., GAT) mass. Adipose tissue mass may be measured by any method known in the art including, but not limited to, weighing the subject, measuring the expression of adipose tissue-specific genes (e.g., PPARγ, SREBP1C, and/or CEBP-alpha) and immunofluorescence staining of adipose tissue-specific proteins (e.g., PPARγ, SREBP1C, and/or CEBP-alpha). Adipose tissue mass may be decreased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, adipose tissue mass is decreased by at least 5%, 10%, 25%, 50%, 75%, 100%, 150%, 200%, 250%, 300%, 350%, 400%, 450%, 500%, 550%, or more relative to a control. A control may be a subject that is not treated with methods provided herein.

An ILC2 and/or a MSC cell may be contacted by more than one RET agonist and/or ADRB2 agonists. In some embodiments, an ILC2 and/or a MSC is contacted by one-ten, two-nine, three-eight, four-seven, or five-six RET/ADRB2 agonists. In some embodiments, an ILC2 and/or an MSC is contacted with one, two, three, four, five, six, seven, eight, nine, ten, or more RET/ADRB2 agonists. In embodiments wherein an ILC2 and/or a MSC is contacted with multiple (e.g., two or more) RET/ADRB2 agonists, the ILC2 and/or MSC may be contacted with the multiple RET/ADRB2 agonists simultaneously or sequentially.

Disorder Associated with Decreased ILC2-Expression or Activity

Also provided herein, in some aspects, are methods of treating a disorder associated with decreased ILC2 activity or proliferation in a subject. Methods of treating a disorder associated with decreased ILC2 activity or proliferation may include contacting an ILC2 (e.g., in a subject) with any RET agonist provided herein, contacting a MSC with any ADRB2 agonist provided herein, or a combination thereof compared to a control. A control may be a subject with ILC2s that are not contacted with a RET agonist, a subject with MSCs that is are contacted with an ADRB2 agonist, or the same subject with an ICL2 before it is contacted with a RET agonist and/or an MSC before it is contacted with an ADRB2 agonist.

A disorder associated with decreased ILC2 activity or proliferation in a subject may be any disorder associated with decreased ILC2 activity or proliferation. Non-limiting examples of disorders associated with decreased ILC2 activity or proliferation include: weight gain, obesity, diabetes, metabolic syndrome, or a combination thereof.

In some embodiments, a disorder associated with decreased ILC2 activity or proliferation is weight gain. Weight gain can occur due to an increase in adipose tissue, body fluid, or muscle mass. An increase in adipose tissue occurs when a subject regularly consumes more calories than are burned through daily physical activity. An increase in body fluid can come from medications, fluid and salt retention, intravenous fluid infusion, kidney failure, or heart failure. An increase in muscle mass is commonly seen when exercising. In some embodiments, a disorder associated with decreased ILC2 activity or proliferation is weight gain due to an increase in adipose tissue (e.g., GAT). Conventional treatment for weight gain (e.g., due to an increase in adipose tissue) includes, but is not limited to, increased daily physical activity, increased consumption of foods low in saturated fat and trans fat, and treatment with diuretics (e.g., furosemide, bumetanide, torsemide, hydrochlorothiazide, metolazone, spironolactone).

In some embodiments, a disorder associated with decreased ILC2 activity or proliferation is obesity. Obesity may be diagnosed by any method and occur with any symptoms described herein.

In some embodiments, a disorder associated with decreased ILC2 activity or proliferation is diabetes. Diabetes is a disorder in which a subject's ability to produce or respond to insulin is impaired, resulting in glucose levels greater than or equal to 200 mg/dL in blood and urine. Diabetes may be Type 1 diabetes (juvenile diabetes) or Type 2 diabetes (adult-onset diabetes). In some embodiments, diabetes is Type 2 diabetes. In Type 2 diabetes, a subject does not produce enough insulin or does not respond to insulin, resulting in hyperglycemia. Symptoms of Type 2 diabetes include, but are not limited to: increased thirst, frequent urination, hunger, fatigue, and blurred vision. Conventional treatment for diabetes includes, but is not limited to, increased daily physical activity, increased consumption of foods low in saturated fat and trans fat, monitoring blood glucose levels, treatment with anti-diabetes medications (e.g., metformin, sulfonylureas, glinides, thiazolidinediones, DDP-4 inhibitors, GLP-1 receptor agonists, SGLT2 inhibitors), and insulin therapy.

In some embodiments, a disorder associated with decreased ILC2 activity or proliferation is metabolic syndrome (also known as insulin resistance syndrome). Metabolic syndrome is a cluster of conditions that increase the risk of heart disease, stroke, and diabetes (e.g., Type 2 diabetes) relative to a subject that does not have metabolic syndrome. Metabolic syndrome is typically diagnosed if a subject has three or more of the following: high blood pressure (e.g., systolic is 135 mm Hg or higher and diastolic is 85 mm Hg or higher), high blood sugar (e.g., greater than or equal to 100 mg/dL fasting glucose), excess body fat around the waist (e.g., waist circumference greater than 40 inches in men and greater than 35 inches in women), and abnormal cholesterol levels (total cholesterol greater than 200 mg/dL, non-high density lipoprotein greater than 130 mg/dL, low density lipoprotein greater than 100 mg/dL, and/or high density lipoprotein less than 50 mg/dL). Conventional treatment for metabolic syndrome includes, but is not limited to: increased daily physical activity, increased consumption of foods low in saturated fat and trans fat, smoking cessation, reducing stress, high blood pressure medication (e.g., ACE inhibitors, angiotensin II receptor blockers, diuretics, beta-blockers), cholesterol medication (e.g., statins, niacin, bile acid resins), diabetes medication (metformin, pioglitazone, rosiglitazone), and low dose aspirin.

ILC2 activity may be measured by any method known in the art including, but not limited to: quantitative PCR measurement and fluorescence quantification of proteins produced by ILC2 cells (e.g., cytokines). ILC2 proliferation may be measured by any method known in the art including, but not limited to: immunohistochemistry of ILC2 surface proteins and quantitative PCR of ILC2-specific proteins (e.g., RET receptor, neuropeptide receptor Nmur1, interleukin-33 receptor ST2, IL-17A/IL-17B receptor).

ILC2 activity may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 activity is increased by at least 5%, 10%, 25%, 50%, 75%, 100%, 150%, 200%, 250%, 300%, 350%, 400%, 450%, 500%, 550%, or more relative to a control. ILC2 proliferation may be increased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 proliferation is increased by at least 5%, 10%, 25%, 50%, 75%, 100%, 150%, 200%, 250%, 300%, 350%, 400%, 450%, 500%, 550%, or more relative to a control. A control may be an ILC2 that is not contacted with a RET agonist or the same ILC2 before it is contacted with a RET agonist; an ILC2 in a cell in which an MSC is not contacted with an ADRB2 agonist or the same ILC2 in a cell before it is contacted with an ADRB2 agonist.

An ILC2 and/or a MSC cell may be contacted by more than one RET agonist and/or ADRB2 agonists. In some embodiments, an ILC2 and/or a MSC is contacted by one-ten, two-nine, three-eight, four-seven, or five-six RET/ADRB2 agonists. In some embodiments, an ILC2 and/or an MSC is contacted with one, two, three, four, five, six, seven, eight, nine, ten, or more RET/ADRB2 agonists. In embodiments wherein an ILC2 and/or a MSC is contacted with multiple (e.g., two or more) RET/ADRB2 agonists, the ILC2 and/or MSC may be contacted with the multiple RET/ADRB2 agonists simultaneously or sequentially.

Disorder Associated with Increased ILC2 Activity or Proliferation

Also provided herein, in some aspects, are methods of treating a disorder associated with increased ILC2 activity or proliferation in a subject. Methods of treating a disorder associated with increased ILC2 activity or proliferation may include contacting an ILC2 (e.g., in a subject) with a RET antagonist, contacting a MSC with a ADRB2 antagonist, or a combination thereof compared to a control. A control may be a subject with ILC2s that are not contacted with a RET antagonist, a subject with MSCs that are contacted with an ADRB2 antagonist, or the same subject with an ICL2 before it is contacted with a RET antagonist and/or an MSC before it is contacted with an ADRB2 antagonist.

A disorder associated with increased ILC2 activity or proliferation in a subject may be any disorder associated with increased ILC2 activity or proliferation. Non-limiting examples of disorders associated with increased ILC2 activity or proliferation include: hypothermia, cachexia, allergy, helminth infection, allergic asthma, atopic dermatitis, intestinal inflammatory disease, or a combination thereof.

In some embodiments, a disorder associated with increased ILC2 activity or proliferation is hypothermia. As used herein, hypothermia means a significant and potentially dangerous drop in body temperature. Normal body temperature in a human subject is around 98.6° F. (37° C.), and hypothermia occurs as body temperature in a human subject falls below 95° F. (35° C.). Hypothermia is often caused by exposure to cold weather or immersion in cold water. Conventional treatment for hypothermia includes methods to warm a subject (e.g., human) back to a normal body temperature.

In some embodiments, a disorder associated with increased ILC2 activity or proliferation is cachexia. Cachexia is loss of more than 5% of body weight over 12 months or less when a subject is not trying to lose weight and has a known illness or disease, along with at least three of: reduced muscle strength, fatigue, appetite loss, low fat-free mass index, elevated inflammation identified by blood tests compared to control, anemia, or low levels of the protein albumin. Cachexia occurs in diseases such as cancer, congestive heart failure, chronic obstructive pulmonary disease (COPD), chronic kidney disease, cystic fibrosis, and rheumatoid arthritis. Conventional treatment to alleviate the symptoms of cachexia include appetite stimulants (e.g., megestrol acetate, Megace); drugs such as dronabinol (Marinol) to improve nausea, appetite, and mood; medications that decrease inflammation; diet changes; nutritional supplements; and adapted exercise.

In some embodiments, a disorder associated with increased ILC2 activity or proliferation is allergy. Allergy is an immune system response to an exogenous substance that is not harmful. Categories of allergies include, but are not limited to: foods (e.g., cow's milk, soy, eggs, wheat, peanuts, tree nuts, fish, shellfish), seasonal (e.g., pollen, mold, ragweed), latex, medications (e.g., penicillin), insect stings or bites (e.g., wasps, bees, hornets, ants, mosquitoes, ticks), and toxins (e.g., poison ivy, eastern poison oak, western poison oak, poison sumac). Symptoms of allergy include, but are not limited to: red eyes, itchy rash, sneezing, runny nose, shortness of breath, swelling, and hives. Conventional treatment to alleviate allergy include medications (e.g., antihistamines, glucocorticoids, epinephrine, mast cell stabilizers, antileukotriene agents, anti-cholinergics, decongestants), immunotherapy (e.g., injection immunotherapy, sublingual immunotherapy), and alternative medicine (e.g., saline nasal irrigation, butterbur).

In some embodiments, a disorder associated with increased ILC2 activity or proliferation is helminth infection. A helminth is a parasitic worm that lives in and feeds on a living host. Categories of helminths include, but are not limited to: annelids (e.g., ringed worms, segmented worms), platyhelminths (e.g., tapeworms, flukes, blood flukes), nematodes (e.g., roundworms), and acanthocephalopods (e.g., thorny-headed worms). Symptoms of helminth infection include, but are not limited to: abdominal pain, weight loss, nausea, vomiting, fever, cough, dyspnea, urticaria, myalgia, pneumonitis, lymphadenopathy, hepatosplenomegaly, and convulsions. Conventional treatment for helminth infection includes: mebendazole, albendazole, niclosamide, praziquantel, and steroids (e.g., dexamethasone, prednisolone).

In some embodiments, a disorder associated with increased ILC2 activity or proliferation is allergic asthma. Allergic asthma is a long-term inflammatory disease of the airways of the lungs. Allergic asthma may occur due to exposure to any known allergen including, but not limited to: dust mites, cockroaches, animal dander, and mold. Non-limiting symptoms of allergic asthma include: wheezing, coughing, chest tightness, and shortness of breath. Conventional treatment for allergic asthma includes: avoiding allergens, inhaled corticosteroids, and, anti-leukotriene agents.

In some embodiments, a disorder associated with increased ILC2 activity or proliferation is atopic dermatitis. Atopic dermatitis (atopic eczema) is long-term inflammation of the skin that results in itchy, red, swollen, and cracked skin. Atopic dermatitis is typically diagnosed when a subject has itchy skin plus three or more of the following: skin creases are involved (e.g., flexural dermatitis of fronts of ankles, antecubital fossae, popliteal fossae, skin around eyes, neck, and cheeks), history of asthma or allergic rhinitis (or family history if subject is less than 4 years old), symptoms beginning before age 2, history of dry skin (within the past year), and dermatitis visible on flexural surfaces or on the cheeks, forehead, and extensor surfaces. Clear fluid may come from the affected skin areas. The cause of atopic dermatitis is unknown, but is thought to involve genetics, immune system dysfunction, environmental exposure, and difficulties with the permeability of the skin. Conventional treatment for atopic dermatitis includes: avoiding triggers (e.g., wool clothing, soaps, perfumes, chlorine, dust, cigarette smoke), daily bathing and applying moisturizing creams afterwards, steroid creams, and drugs that alleviate itching.

In some embodiments, a disorder associated with increased ILC2 activity or proliferation is intestinal inflammatory disease. Intestinal inflammatory disease (also known as inflammatory bowel disease (IBD)) is ongoing inflammation of all or part of the intestinal tract. Intestinal inflammatory disease may occur in the small intestine or the large intestine (bowel). Intestinal inflammatory disease is an umbrella term that covers any intestinal inflammatory disease including, but not limited to: ulcerative colitis (UC) and Crohn's disease (CD). Symptoms of intestinal inflammatory disease include diarrhea, fatigue, abdominal pain and cramping, blood in stool, reduced appetite, and unintended weight loss.

Conventional treatment for intestinal inflammatory disease includes, but is not limited to: anti-inflammatory drugs (e.g., corticosteroids, aminosalicylates), immune system suppressors (e.g., azathioprine, mercaptopurine, methotrexate), biologics (e.g., infliximab, adalimumab, golimumab, certolizumab, vedolizumab, ustekinumab), antibiotics (e.g., ciprofloxacin, metronidazole), anti-diarrheals (e.g., psyllium powder, methylcellulose, loperamide), pain relievers (e.g., acetaminophen, ibuprofen, naproxen sodium, diclofenac sodium), and vitamins.

A RET antagonist may be any RET antagonist known in the art. Antagonists of RET include peptide antagonists (including modified peptides and conjugates), inhibitory antibody molecules, inhibitory nucleic acid molecules, and small molecules. Some of the RET antagonists may be entirely specific for RET, may antagonize RET preferentially (as compared to other tyrosine kinases), or may antagonize both RET and other tyrosine kinases (such as some of the small molecule RET tyrosine kinase inhibitors described below. Such antagonists may be useful even if RET is antagonized less than other tyrosine kinases, but it is preferred that the antagonists used in the methods described herein antagonize RET to a greater extent than other tyrosine kinases. As used herein, antagonizing RET preferentially (as compared to other tyrosine kinases) means that the antagonist antagonizes RET at least 10%, 25%, 50%, 100%, 200%, 300%, 400%, 500%, 600%, 700%, 800%, 900%, 1000%, or more than other tyrosine kinases.

Antagonists of RET include antibodies that specifically bind and inhibit: (a) RET tyrosine kinase activity, (b) a GDNF Family binding Receptor alpha (GFRα), or (c) a GFRα ligand, or an antigen-binding fragment thereof. Examples include the antibodies described in U.S. Pat. Nos. 8,968,736, 9,522,185, and US 2017/0096488 that bind human GFRα3. RET-binding antibodies are known in the art, such as those described in U.S. Pat. No. 6,861,509, and various commercially-available antibodies. Antibodies that specifically bind to and inhibit: (a) RET tyrosine kinase activity, (b) a GDNF Family binding Receptor alpha (GFRα), or (c) a GFRα ligand, can be obtained by screening for one of these activities among a set of antibodies binding to RET, a GFRα, or a GFRα ligand.

Antagonists of RET include an inhibitory nucleic acid molecule that reduces expression, transcription or translation of RET, a GFRα, or a GFRα ligand. Suitable inhibitory nucleic acid molecules include: RET-specific, a GFRα-specific, or a GFRα ligand-specific inhibitory nucleic acid, e.g., an siRNA, antisense, aptamer, or ribozyme targeted specifically to RET, a GFRα, or a GFRα ligand.

Antagonists of RET include a RET tyrosine kinase inhibitor. Exemplary RET tyrosine kinase inhibitors include AST 487, motesanib, cabozantinib, vandetanib, ponatinib, sunitinib, sorafenib, and alectinib.

AST 487 (also known as NVP-AST487; 630124-46-8; UNII-W34UO2M4T6); IUPAC name: 1-[4-[(4-ethylpiperazin-1-yl)methyl]-3-(trifluoromethyl)phenyl]-3-[4-[6-(methylamino)pyrimidin-4-yl]oxyphenyl]urea) is an inhibitor of RET, receptor-type tyrosine-protein kinase FLT3, Kinase Insert Domain Receptor (KDR; VEGFR2), Abelson murine leukemia viral oncogene homolog 1 (c-ABL), and stem cell factor receptor (c-KIT) that has been shown to inhibit RET autophosphorylation and activation of downstream effectors (Akeno-Stuart et al., Cancer Res. 2007 Jul. 15; 67(14):6956-64). A chemical structure of AST 487 is shown below:

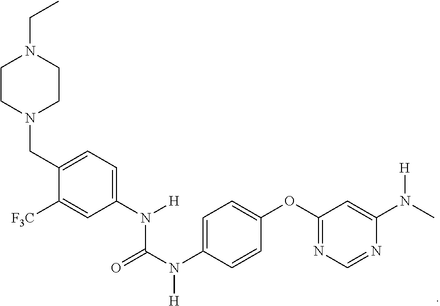 [SMILES: CCN1CCN(Cc2ccc(NC(=O)Nc3ccc(Oc4cc(NC)ncn4)cc3)cc2C(F)(F)F)CC1]

Motesanib (also known as AMG-706; IUPAC name: N-(3,3-dimethyl-2,3-dihydro-1H-indol-6-yl)-2-[(pyridin-4-ylmethyl)amino]pyridine-3-carboxamide) is an inhibitor of RET, VEGFRs, platelet-derived growth factor receptors (PDGFRs), and c-KIT. A chemical structure of motesanib is shown below:

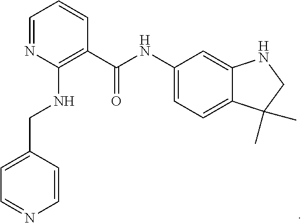 [SMILES: CC1(C)CNc2cc(NC(=O)c3cccnc3NCc3ccncc3)ccc21]

Cabozantinib (also known as CABOMETYX; COMETRIQ; XL-184; BMS-907351; IUPAC name: N-(4-((6,7-dimethoxyquinolin-4-yl)oxy)phenyl)-N′-(4-fluorophenyl)cyclopropane-1,1-dicarboxamide) is an inhibitor of RET, hepatocyte growth factor receptor (MET), AXL receptor tyrosine kinase (AXL; tyrosine-protein kinase receptor UFO) and vascular endothelial growth factor receptor receptors (VEGFR) including VEGFR2. A chemical structure of cabozantinib is shown below:

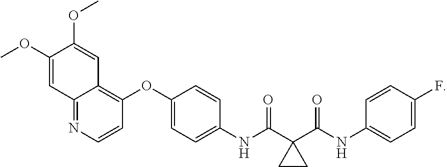 [SMILES: COc1cc2nccc(Oc3ccc(NC(=O)C4(C(=O)Nc5ccc(F)cc5)CC4)cc3)c2cc1OC]

Vandetanib (also known as CAPRELSA; ZACTIMA; ZD-6474; IUPAC name: N-(4-bromo-2-fluorophenyl)-6-methoxy-7-((1-methylpiperidin-4-yl)methoxy)quinazolin-4-amine) is an inhibitor of RET, VEGFRs including VEGFR2, and epidermal growth factor receptor (EGFR). A chemical structure of vandetanib is shown below:

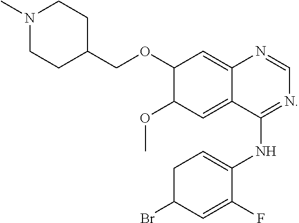 [SMILES: COC1C=c2c(NC3=CCC(Br)C=C3F)ncnc2=CC1OCC1CCN(C)CC1]

Ponatinib (also known as ICLUSIG; AP24534; IUPAC name: 3-(2-Imidazo[1,2-b]pyridazin-3-ylethynyl)-4-methyl-N-[4-[(4-methylpiperazin-1-yl)methyl]-3-(trifluoromethyl)phenyl]benzamide) is an inhibitor of RET and fibroblast growth factor receptor (FGFR). A chemical structure of ponatinib is shown below:

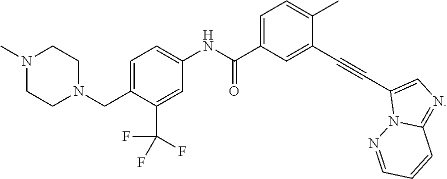 [SMILES: Cc1ccc(C(=O)Nc2ccc(CN3CCN(C)CC3)c(C(F)(F)F)c2)cc1C#Cc1cnc2cccnn12]

Sunitinib (also known as SUTENT; SU11248; IUPAC name: N-(2-diethylaminoethyl)-5-[(Z)-(5-fluoro-2-oxo-1H-indol-3-ylidene)methyl]-2,4-dimethyl-1H-pyrrole-3-carboxamide) is an inhibitor of RET, PGFRs, VEGFRs, c-KIT, granulocyte colony-stimulating factor receptor (GCSFR) and FLT3. A chemical structure of sunitinib is shown below:

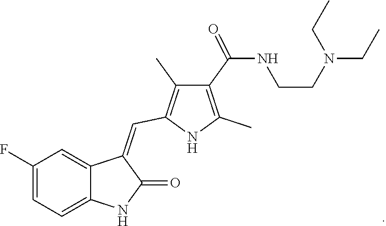 [SMILES: CCN(CC)CCNC(=O)c1c(C)[nH]c(/C=C2\C(=O)Nc3ccc(F)cc32)c1C]

Sorafenib (also known as NEXAVAR; IUPAC name: 4-[4-[[4-chloro-3-(trifluoromethyl)phenyl]carbamoylamino] phenoxy]-N-methyl-pyridine-2-carboxamide) is an inhibitor of RET, VEGFR, PDGFR and Raf family kinases. A chemical structure of sorafenib is shown below:

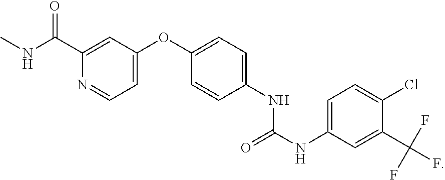 [SMILES: CNC(=O)c1cc(Oc2ccc(NC(=O)Nc3ccc(Cl)c(C(F)(F)F)c3)cc2)ccn1]

Alectinib (also known as ALECENSA; IUPAC name: 9-ethyl-6,6-dimethyl-8-[4-(morpholin-4-yl)piperidin-1-yl]-11-oxo-6,11-dihydro-5H-benzo[b]carbazole-3-carbonitrile) is an inhibitor of RET, and anaplastic lymphoma kinase (ALK). A chemical structure of alectinib is shown below:

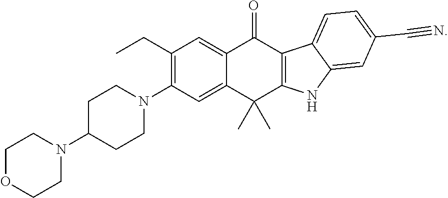 [SMILES: CCc1cc2c(cc1N1CCC(N3CCOCC3)CC1)C(C)(C)c1[nH]c3cc(C#N)ccc3c1C2=O]

Other suitable RET antagonists include the molecules described in: U.S. Pat. Nos. 6,235,769, 7,504,509, 8,067,434, 8,426,437, 8,629,135, 8,937,071, 8,999,973, 9,035,063, 9,382,238, 9,297,011, US 2015/0238477, US 2015/0272958, US 2016/0271123, US 20160354377, US 2017/0096425, and US 2017/0121312, and related patent applications worldwide.

An ADRB2 antagonist may be any ADRB2 antagonist known in the art. An ADRB2 agonist may be a non-selective beta-adrenergic receptor antagonist that binds both beta-1 and beta-2 adrenergic receptors and include: propranolol, bucindolol, carteolol, carvedilol, labetalol, nadolol, oxprenolol, penbutolol, pindolol, sotalol, and timolol. An ADRB2 antagonist may be specific for ADRB2 (compared to other beta-adrenergic receptors). Non-limiting examples of ADRB2-specific antagonists include: butaxamine and ICI-118,551. Other possible ADRB2 antagonists for use in a method herein include: acebutolol, atenolol, betaxolol, bisoprolol, celiprolol, metoprolol, nebivolol, esmolol, and SR 59230A.

ILC2 activity may be measured by any method known in the art including, but not limited to: quantitative PCR measurement and fluorescence quantification of proteins produced by ILC2 cells (e.g., cytokines). ILC2 proliferation may be measured by any method known in the art including, but not limited to: immunohistochemistry of ILC2 surface proteins and quantitative PCR of ILC2-specific proteins (e.g., RET receptor, neuropeptide receptor Nmur1, interleukin-33 receptor ST2, IL-17A/IL-17B receptor).

ILC2 activity may be decreased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 activity is decreased by at least 5%, 10%, 25%, 50%, 75%, 100%, 150%, 200%, 250%, 300%, 350%, 400%, 450%, 500%, 550%, or more relative to a control. ILC2 proliferation may be decreased by 5%-50%, 10%-100%, 25%-150%, 50%-200%, 75%-250%, 100%-300%, 150%-350%, 200%-400%, 250%-450%, 300%-500%, 350%-550% or more relative to a control. In some embodiments, ILC2 proliferation is decreased by at least 5%, 10%, 25%, 50%, 75%, 100%, 150%, 200%, 250%, 300%, 350%, 400%, 450%, 500%, 550%, or more relative to a control. A control may be an ILC2 that is not contacted with a RET antagonist or the same ILC2 before it is contacted with a RET antagonist; an ILC2 in a cell in which an MSC is not contacted with an ADRB2 antagonist or the same ILC2 in a cell before it is contacted with an ADRB2 antagonist.

An ILC2 and/or a MSC cell may be contacted by more than one RET antagonist and/or ADRB2 antagonists. In some embodiments, an ILC2 and/or a MSC is contacted by one-ten, two-nine, three-eight, four-seven, or five-six RET/ADRB2 antagonists. In some embodiments, an ILC2 and/or an MSC is contacted with one, two, three, four, five, six, seven, eight, nine, ten, or more RET/ADRB2 antagonists. In embodiments wherein an ILC2 and/or a MSC is contacted with multiple (e.g., two or more) RET/ADRB2 antagonists, the ILC2 and/or MSC may be contacted with the multiple RET/ADRB2 antagonists simultaneously or sequentially.

### Cold Exposure

Also provided herein, in some aspects, are methods of treating cold exposure. Methods of treating cold exposure may include contacting an ILC2 (e.g., in a subject) with a RET agonist, contacting a MSC with an ADRB2 agonist, or a combination thereof compared to a control. A control may be a subject with ILC2s that are not contacted with a RET agonist, a subject with MSCs that are contacted with an ADRB2 agonist, or the same subject with an ICL2 before it is contacted with a RET agonist and/or an MSC before it is contacted with an ADRB2 agonist.

Cold exposure can occur outdoors in wet, windy, and/or cold weather or indoors in a dwelling that is not sufficiently heated to prevent cold exposure. If left untreated, cold exposure may result in an injury including, but not limited to: frostnip, frostbite, trench foot, chilblains, and hypothermia. Frostnip causes numbness or blue-white skin for a short time, but normal feeling and color returns upon warming. Frostbite is freezing of the skin and tissues under the skin and does not return to normal feeling or color upon warming. Trench foot is an injury that occurs gradually over several days of exposure to cold temperatures where the skin does not actually freeze and is characterized by red skin, numbness or burning pain, leg cramps, and development of blisters or ulcers after 2 to 7 days. Chilblains (perniosis) is a reaction to cold temperatures and are characterized by local redness and swelling, skin bumps, changes in sensation, tender blue bumps that develop after rewarming, and blisters and ulcers. Hypothermia is discussed above.

Risk factors for cold exposure injury include, but are not limited to: being an infant (<than 1 year old); being an older adult (>65 years old); consuming alcohol, being outdoors at high altitudes, in windy, wet weather or immersed in cold water; being tired or dehydrated, being exposed to cold temperatures in a workplace; having conditions such as diabetes, HIV, cancer, or heart disease; taking certain medications such as anticoagulants, immunosuppressants; and having recent health events such as surgery or injury.

A subject's temperature may be increased by a method of treating cold exposure provided herein by 1° F.-45° F., 5° F.-40° F., 10° F.-35° F., 15° F.-30° F., 20° F.-25T, or 1° C.-25° C., 5° C.-20° C., 10° C.-15° C. or more relative to a control. In some embodiments, a subject's temperature is increased by at least IT, 2° F., 3° F., 4° F., 5° F., 6° F., 7° F., 8° F., 9° F., 10° F., 1IT, 12° F., 13° F., 14° F., 15° F., 16° F., 17° F., 18° F., 19° F., 20° F., 21° F., 22° F., 23° F., 24° F., 25° F., 26° F., 27° F., 28° F., 29° F., 30° F., 31° F., 32° F., 33° F., 34° F., 35° F., 36° F., 37° F., 38° F., 39° F., 40° F., 41° F., 42° F., 43° F., 44° F., 45° F., or more compared to control. In some embodiments, a subject's temperature is increased by at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. A control may be an ILC2 that is not contacted with a RET agonist or the same ILC2 before it is contacted with a RET agonist; an ILC2 in a cell in which an MSC is not contacted with an ADRB2 agonist or the same ILC2 in a cell before it is contacted with an ADRB2 agonist.

An ILC2 and/or a MSC cell may be contacted by more than one RET agonist and/or ADRB2 agonists. In some embodiments, an ILC2 and/or a MSC is contacted by one-ten, two-nine, three-eight, four-seven, or five-six RET/ADRB2 agonists. In some embodiments, an ILC2 and/or an MSC is contacted with one, two, three, four, five, six, seven, eight, nine, ten, or more RET/ADRB2 agonists. In embodiments wherein an ILC2 and/or a MSC is contacted with multiple (e.g., two or more) RET/ADRB2 agonists, the ILC2 and/or MSC may be contacted with the multiple RET/ADRB2 agonists simultaneously or sequentially.

### Methods of Administration

In some aspects, methods provided herein comprise contacting a cell (e.g., ILC2, MSC) with a RET agonist, a RET antagonist, an ADRB2 agonist, or an ADRB2 antagonist. Contacting means providing a compound (e.g., RET agonist, RET antagonist, ADRB2 agonist, ADRB2 antagonist) or pharmaceutical composition comprising a compound to a cell.

A cell contacted in a method provided herein may be in vitro or in vivo. In some embodiments, a cell contacted in a method provided herein is in vitro. An in vitro cell may be maintained under conditions that mimic an in vivo environment (e.g., in cell culture). An in vitro cell may be single or part of a population of cells. A population of cells may comprise 2 cells-1,000 cells, 500 cells-10,000 cells, 5,000 cells-100,000 cells, 50,000 cells-1,000,000 cells, 500,000 cells-10,000,000 cells, or more.

In some embodiments, a cell contacted in a method provided herein is in vivo. When the cell is contacted in vivo, a RET agonist, a RET antagonist, an ADRB2 agonist, an ADRB2 antagonist, or a combination thereof may be administered to a subject. A subject may be any subject in need thereof including, but not limited to: a human, a rodent (e.g., mouse, rat, hamster), a non-human primate (e.g., chimpanzee, gorilla, orangutan), a domestic pet (e.g., dog, cat, rabbit), or a livestock animal (e.g., horse, cow, chicken, pig, goat, sheep, donkey). In some embodiments, a subject is a human.

In some embodiments, an in vitro cell (e.g., ILC2s, MSC) is derived from a tissue in a subject. A tissue in a subject may be any tissue occurring in a subject. Derived from a tissue refers to isolation of an in vitro cell from a tissue. Deriving an in vitro cell from a tissue may be using any method known in the art including, but not limited to: chemical digestion (e.g., trypsin) or mechanical tissue digestion (e.g., homogenization). Non-limiting examples of possible tissues from which an in vitro cell may be derived include: adipose, skeletal muscle, smooth muscle, cardiac muscle, nervous, blood, renal, pancreas, stomach, small intestine, large intestine, rectum, brain, spinal cord, bone, cartilage, skin, hair, liver, ovary, uterus, testicular, prostate, cardiac, lung, tracheal, tongue, and salivary gland. In some embodiments, an in vitro cell is derived from an adipose tissue.

In some embodiments, an agonist (e.g., a RET agonist, an ADRB2 agonist), an antagonist (e.g., a RET antagonist, an ADRB2 antagonist), or a combination thereof is administered to a subject in need thereof. Administration may be by any method known in the art including, but not limited to: injection (e.g., intravenous, intramuscular, intraarterial, intraventricular), inhalation, and ingestion (e.g., oral, rectal).

When an agonist (e.g., a RET agonist, an ADRB2 agonist), an antagonist (e.g., a RET antagonist, an ADRB2 antagonist), or a combination thereof is administered to a subject, a dose of each of the agonist and/or antagonists is administered. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment. Multiple doses may also be administered to a subject in need thereof.

The active agents of the invention (e.g., the compounds and cells described herein) are administered to the subject in an effective amount for treating disease. According to some aspects of the invention, an effective amount is that amount, depending on the disease being treated, of an agonist (e.g., a RET agonist, an ADRB2 agonist), an antagonist (e.g., a RET antagonist, an ADRB2 antagonist) alone or in combination with another medicament, which when combined or co-administered or administered alone, results in a therapeutic response to the disease. The biological effect may be the amelioration and or absolute elimination of disease, or of symptoms resulting from the disease. In another embodiment, the biological effect is the complete abrogation of the disease, as evidenced for example, by the absence of a symptom of the disease.

The effective amount of a compound (i.e., any of the agonists, antagonists, or combination thereof) used in methods of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention using routine and accepted methods known in the art, without necessitating undue experimentation. Combined with the teachings provided herein, by choosing among the various active compounds and weighing factors such as potency, relative bioavailability, patient body weight, severity of adverse side-effects and preferred mode of administration, an effective therapeutic treatment regimen can be planned which does not cause substantial toxicity and yet is effective to treat the particular subject.

In other embodiments the compounds may be isolated. As used herein, isolated means that the referenced material is removed from its native environment, e.g., a cell. Thus, an isolated biological material can be free of some or all cellular components, i.e., components of the cells in which the native material is occurs naturally (e.g., cytoplasmic or membrane components). In the case of nucleic acid molecules, an isolated nucleic acid includes a PCR product, an isolated RNA, a synthetically (e.g., chemically) produced RNA, such as an siRNA, an antisense nucleic acid, an aptamer, etc. Isolated nucleic acid molecules include sequences inserted into plasmids, cosmids, or other vectors to form part of a chimeric recombinant nucleic acid construct, or produced by expression of a nucleic acid encoding it. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein, or may be synthetically (e.g., chemically) produced, or produced by expression of a nucleic acid encoding it. An isolated cell, such as an ILC2 cell or an MSC, can be removed from the anatomical site in which it is found in an organism, or may be produced by in vitro expansion of an isolated cell or cell population. An isolated material may be, but need not be, purified.

Purified refers to a protein, a nucleic acid, or a cell or cell population, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified RET agonist, RET antagonist, ADRB2 agonist, ADRB2 antagonist, or a combination thereof is at least 60%, at least 80%, or at least 90% of total protein or nucleic acid or cell population, as the case may be, by weight. In a specific embodiment, a purified RET agonist, RET antagonist, ADRB2 agonist, ADRB2 antagonist, or a combination thereof is purified to homogeneity as assayed by standard, relevant laboratory protocols.

In some embodiments, an agonist (e.g., a RET agonist, an ADRB2 agonist), an antagonist (e.g., a RET antagonist, an ADRB2 antagonist), or a combination thereof is administered to a subject in a pharmaceutical composition. A pharmaceutical composition is sterile in some embodiments. Pharmaceutical compositions of the present invention comprise an effective amount of one or more agents, dissolved or dispersed in a pharmaceutically acceptable carrier. Pharmaceutical or pharmacologically acceptable refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by relevant government regulatory agencies. The compounds are generally suitable for administration to humans. This term requires that a compound or composition be nontoxic and sufficiently pure so that no further manipulation of the compound or composition is needed prior to administration to humans.

A pharmaceutically acceptable carrier includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

### EXAMPLES

### Example 1: Neuro-Mesenchyme Signals Regulate GAT ILC2 Cells

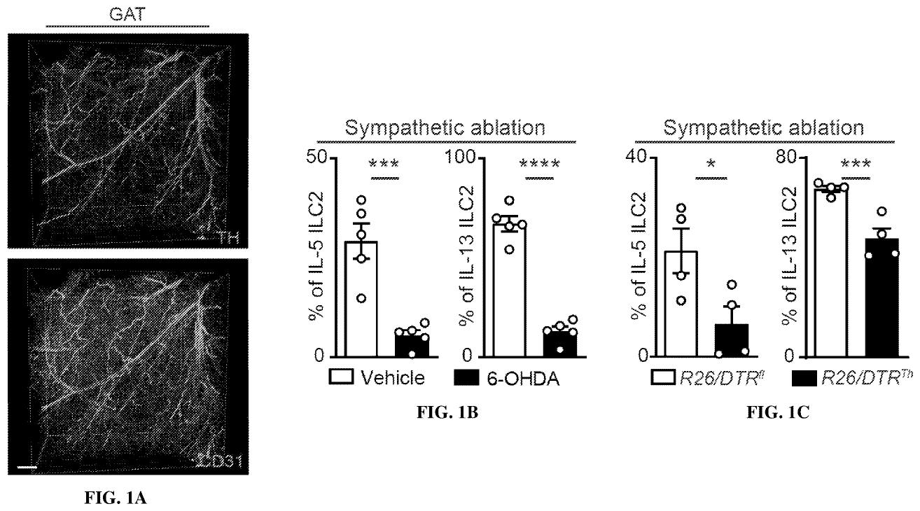

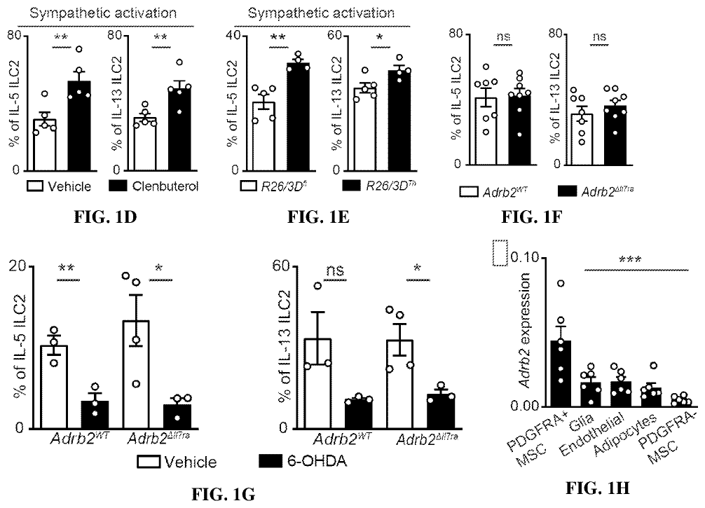

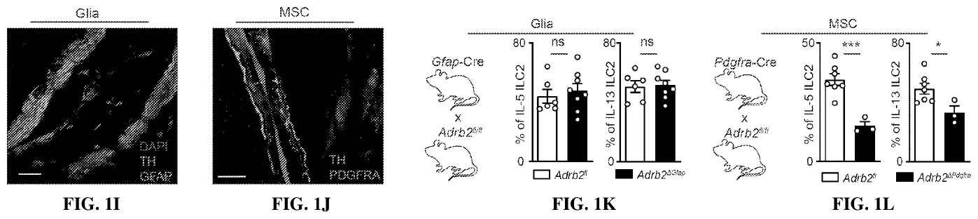

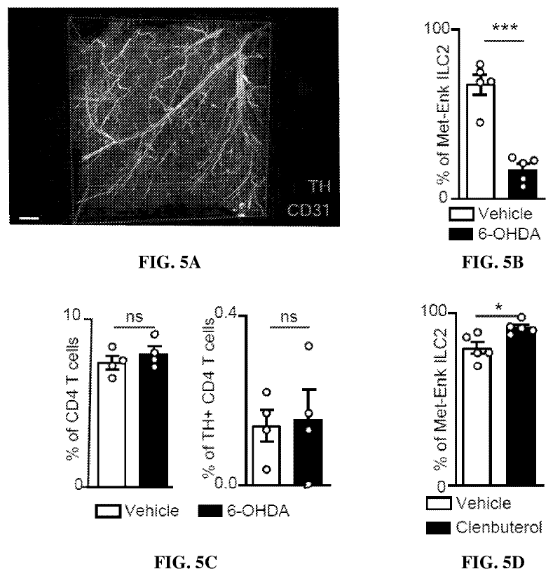

Analysis of the gonadal adipose tissue (GAT) of mice revealed the presence of a dense network of sympathetic neuronal fibers (FIGS. 1A, 5A). To interrogate whether adrenergic cues impact local ILC2 cells (also referred to as “ILC2” and “ILC2s”), dopaminergic and noradrenergic neurons were eliminated using 6-hydroxydopamine (6-OHDA). Systemic ablation of these neurons resulted in a pronounced reduction of ILC2-derived interleukin (IL-) 5, IL-13 and Met-Enk (FIGS. 1B, 5B) and no change in the percentage of CD4 T cells (FIG. 5C). Sequentially, peripheral sympathetic neurons were selectively ablated by breeding ROSA26.DTR (diphtheria toxin receptor) mice to tyrosine hydroxylase-Cre (Th-Cre, R26/DTR^{Th}) mice followed by administration of pegylated diphtheria toxin (PegDTR) to the resulting R26/DTR^{Th} animals¹⁴. Selective ablation of peripheral sympathetic neurons resulted in impaired ILC2 activity (FIG. 1C). In contrast, systemic activation of the beta-2 adrenergic receptor (ADRB2) with clenbuterol led to increased ILC2-derived cytokines in the GAT (FIGS. 1D, 5D). In agreement, neuronal chemogenetic activation in mice carrying designer receptor exclusively activated by designer drugs (DREADD) on sympathetic neurons (R26/3D^{Th}) led to increased ILC2 function (FIG. 1E). To interrogate how the sympathetic tone regulates fat ILC2, Adrb2 was deleted in lymphoid cells by breeding Il7ra-Cre mice to Adrb2^{fl/fl} mice (Adrb2^{ΔIl7ra}). Adipose ILC2 function was unperturbed in Adrb2^{ΔIl7ra} mice (FIG. 1F), suggesting that sympathetic cues regulate adipose ILC2 indirectly. Supporting this hypothesis, chemical sympathetic ablation in Adrb2^{ΔIl7ra} mice still impaired ILC2 cytokine production (FIG. 1G). To elucidate the cellular link between sympathetic neuronal cues and ILC2 activity, the expression of Adrb2 in non-immune adipose-resident cell types was examined. Mesenchymal stromal cells (MSC) that express platelet-derived growth factor receptor alpha (PDGFRA) displayed the highest level of Adrb2 followed by glial cells, endothelial cells, adipocytes and other mesenchymal counterparts (FIG. 1H). Interestingly, glial cells and MSC are in close proximity to GAT sympathetic neurons (FIGS. 11, 1J). Thus, the expression of ADRB2 was disrupted in glial cells and MSC by breeding Gfap- or Pdgfra-Cre mice to Adrb2^{fl/fl} mice (Adrb2^{ΔGfap} and Adrb2^{ΔPdgfra}, respectively). While mice with a glial-autonomous deletion of ADRB2 had unperturbed ILC2 function, Adrb2^{ΔPdgfra} mice displayed reduced fat ILC2-derived cytokines when compared to their littermate controls (FIGS. 1K, 1L). Taken together, these data indicate that neuro-mesenchyme signals regulate GAT ILC2.

### Example 2: Neuro-Mesenchyme Interactions Orchestrate Fat ILC2 Via the Neurotrophic Factor Receptor RET

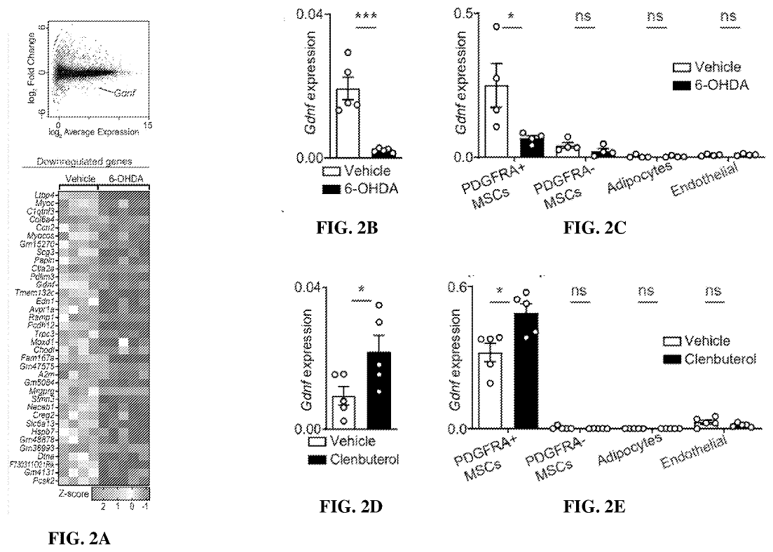

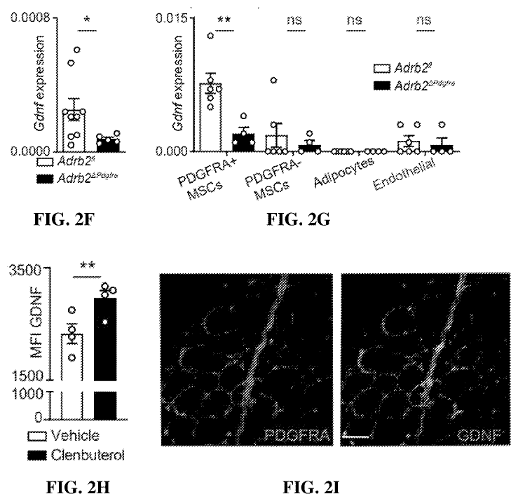

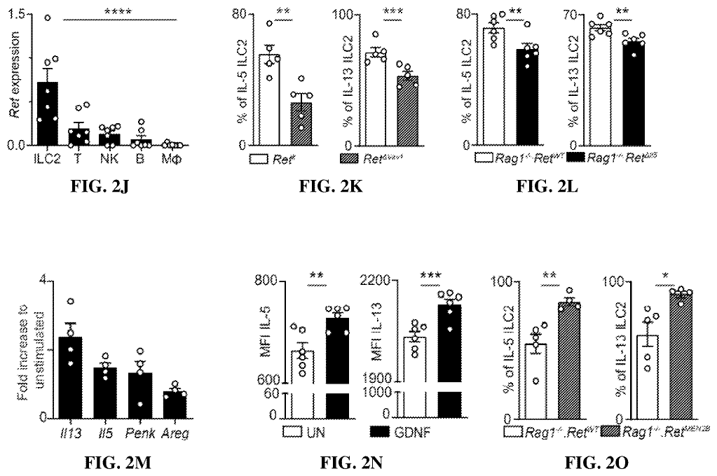

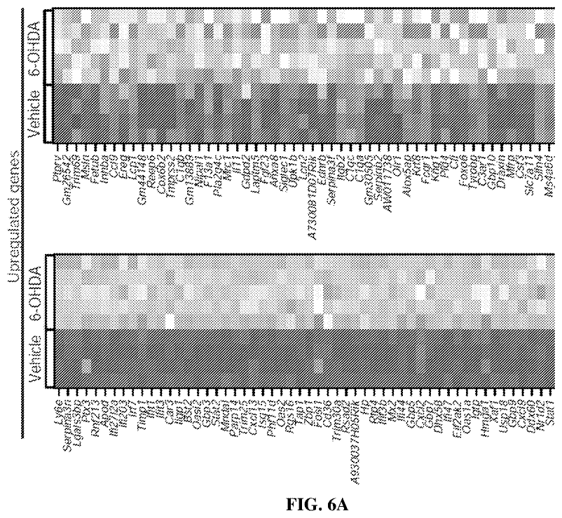

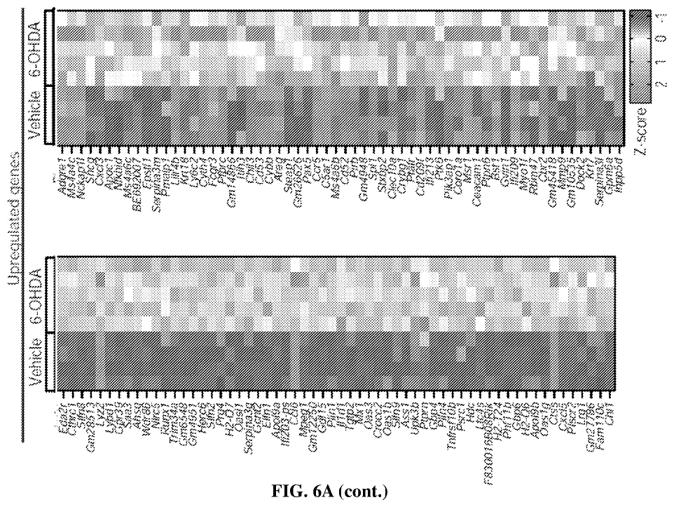

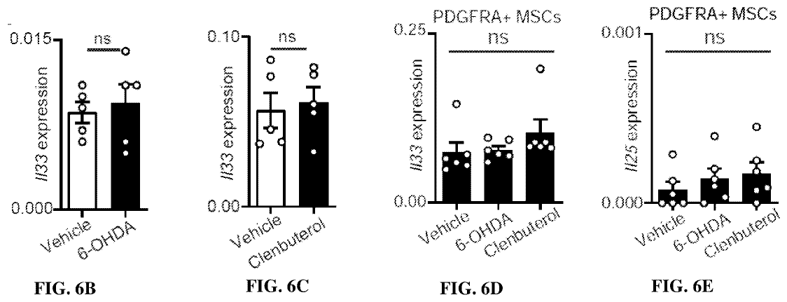

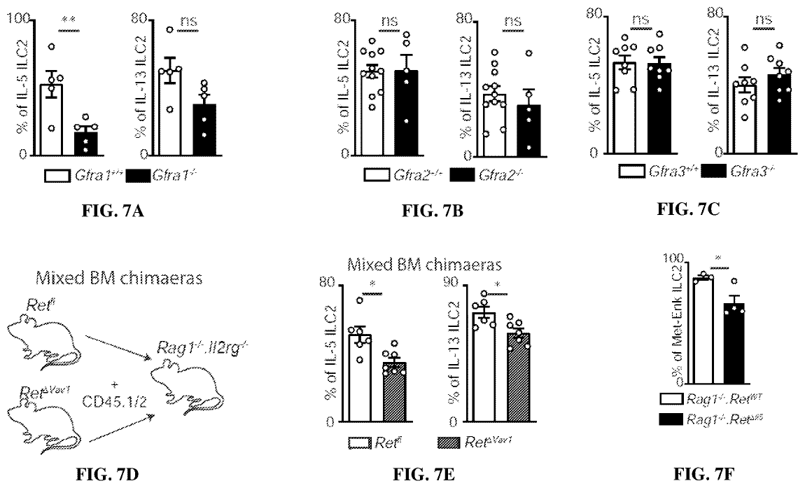

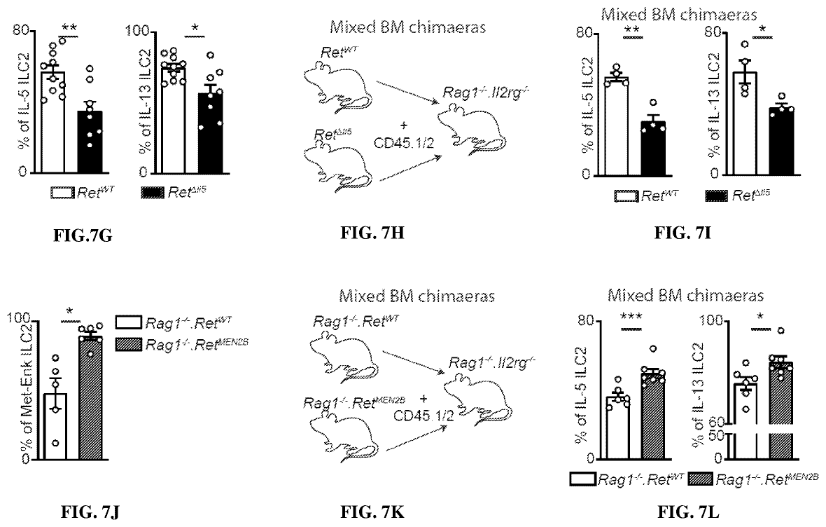

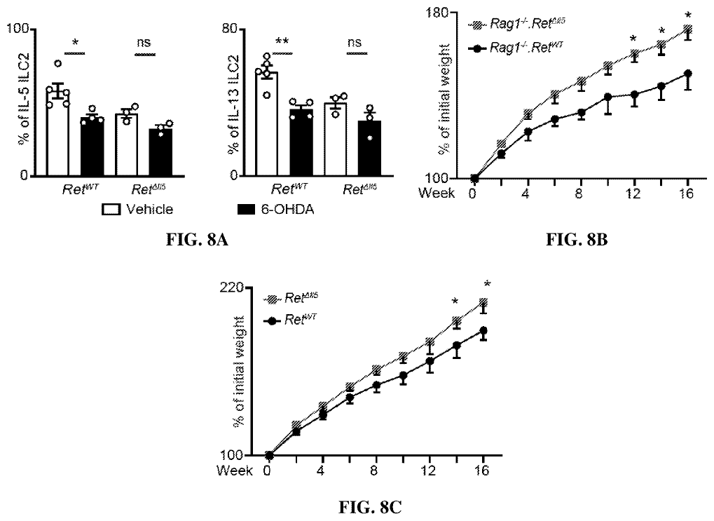

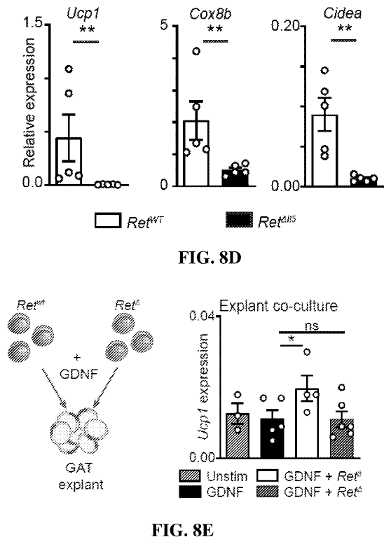

To examine how neuro-mesenchyme interactions lead to activation of GAT-resident ILC2, genome-wide transcriptional profiling of PDGFRA positive MSC from 6-OHDA-treated mice were compared to their littermate controls. This analysis revealed 227 modulated transcripts, of which 35 were downregulated (FIGS. 2A, 6A). Of note, expression of the ILC2-activating alarmins IL-33 and IL-25 was unperturbed in mice treated with either 6-OHDA or clenbuterol (FIGS. 2A, 6A-6E). Amongst the altered genes, the glial-derived neurotrophic factor (GDNF) was highly expressed by PDGFRA positive MSC, and 6-OHDA treatment significantly reduced its expression (FIG. 2A). Importantly, upon chemical sympathetic ablation or ADRB2 stimulation, Gdnf was modulated in the GAT and selectively in PDGFRA+MSC, while unperturbed in other mesenchymal counterparts, adipocytes and endothelial cells (FIGS. 2B-2E). In agreement, GAT and MSC purified from Adrb2^{ΔPdgfra} had reduced Gdnf expression (FIGS. 2F, 2G). In line with these findings, stimulation of ADRB2 in purified MSC led to increased MSC-derived GDNF and PDGFRA+GAT cells colocalised with GDNF^{15,16} (FIGS. 2H, 2I). GDNF family ligands and their preferred coreceptor (GFRa) were shown to activate the tyrosine kinase receptor rearranged during transfection (RET) in the nervous system, kidney and subsets of haematopoietic cells¹⁶⁻²⁰. Analysis of GAT immune cell subsets revealed that GAT ILC2 express high levels of Ret (FIG. 2J). To explore the role of the kinase receptor RET in adipose ILC2, initially Ret was deleted in haematopoietic cells by breeding Vav1-Cre to Ret^{fl/fl} mice (Ret^{ΔVav1}). Ret^{ΔVav1} mice displayed reduced ILC2-derived IL-5, and IL-13 in the GAT (FIG. 2K). In line with this finding, analysis of RET co-receptor single knockouts revealed that Gfra1, the preferential GDNF coreceptor, was selectively required for ILC2 function (FIGS. 7A-7C). Sequentially, mixed bone marrow (BM) chimaeras were utilized by transferring Ret competent (Rett) or deficient (Ret^{ΔVav1}) BM against a third-part wild type competitor into alymphoid hosts (FIG. 7D). Analysis of these chimeric mice suggested that cell intrinsic RET signals are required for innate type 2 cytokines in the GAT (FIG. 7E). To further confirm ILC2-autonomous effects of RET, Il5-Cre mice were bred to Ret^{fl/fl} mice (Ret^{ΔIl5}), and Ret^{ΔIl5} mice were subsequently bred to Rag1^{-/-} mice to exclude putative T helper cell effects. Analysis of Rag1^{-/-}Ret^{ΔIl5} mice confirmed that RET operates in an ILC2-autonomous manner to control innate type 2 cytokines and Met-Enk in the GAT (FIGS. 2L, 7F, 7I). To further evaluate ILC2 activity in mice, mixed bone marrow (BM) chimeras were produced of Ret^{WT} and Ret^{ΔIl5} mice and Rag1^{-/-}, Il2rg^{-/-} knock-out mice (FIG. 7H). Ret^{ΔIl5}Rag1^{-/-}, Il2rg^{-/-} mice had reduced ILC2 activity compared with Ret^{WT}Rag1^{-/-}, Il2rg^{-/-} mice, as demonstrated by reduced IL-5 and IL-13 production (FIG. 7I). In agreement, in vitro activation of purified ILC2 with GDNF family ligands resulted in increased innate cytokine production and analysis of gain-of-function Ret^{MEN2B} bone marrow chimaeras revealed increased ILC2-derived IL-5, IL-13 and Met-Enk (FIGS. 2M-20, 7J-7L). Finally, chemical sympathetic ablation in Ret^{ΔIl5} mice resulted in unperturbed ILC2 cytokine production, indicating that ILC2-autonomous RET signals are required to integrate the sympathetic tone (FIG. 8A). Altogether, these data indicate that neuro-mesenchyme interactions orchestrate fat ILC2 via the neurotrophic factor receptor RET.

### Example 3: ILC2-Intrinsic Neurotrophic Factor Cues are Required to Control Adipose Homeostasis and Obesity

To determine whether ILC2-intrinsic RET signals regulate adipose tissue physiology, varying degrees of RET signals were tested to set the propensity to obesity and associated glucose tolerance dysfunction²¹. Initially, Ret^{ΔVav1} mice were fed with high-fat diet (HFD).

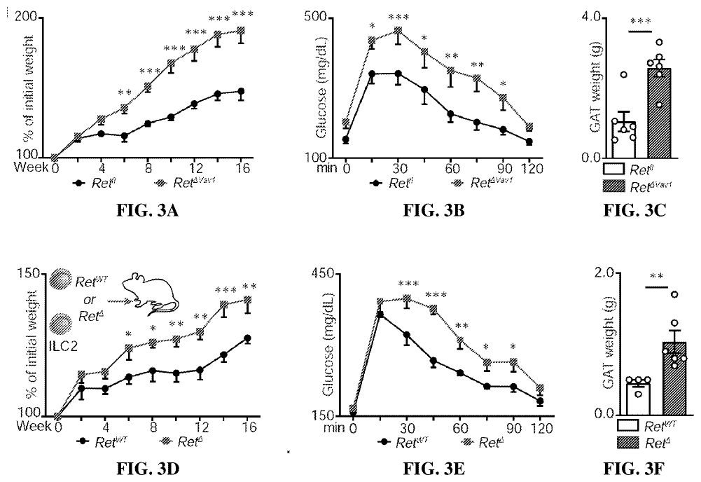

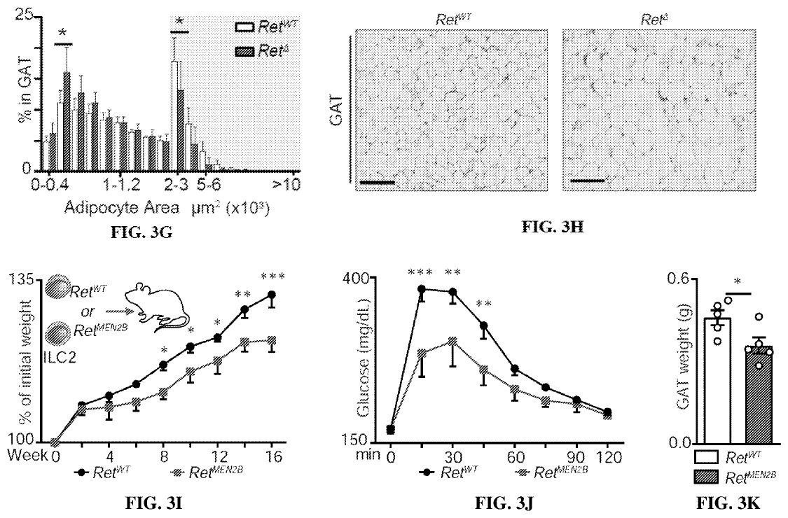

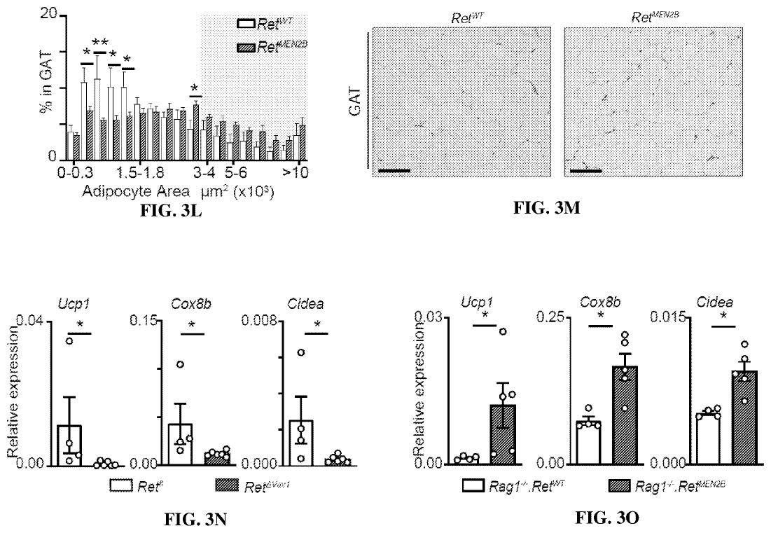

When compared to their littermate controls, Ret^{ΔVav1} and Ret^{ΔIl5} mice had increased susceptibility to HFD-induced obesity, decreased glucose tolerance and increased GAT weight (FIGS. 3A-3C, 8B, 8C). To more specifically define the link between ILC2, GDNF-RET signalling and propensity to obesity, chimaeras of RET-sufficient and RET-deficient ILC2 were generated in alymphoid host mice. RET-deficient ILC2-chimaeras had increased susceptibility to HFD-induced obesity, decreased glucose tolerance and altered frequencies of adipocyte sizes (FIGS. 3D-3H). In contrast, chimaeras generated with ILC2 from gain-of-function Ret^{MEN2B} mice displayed resistance to HFD-induced obesity, improved glucose tolerance and increased frequency of small size adipocytes (FIGS. 3I-3M). Type 2 cytokines and Met-Enk have been shown to promote energy expenditure through adipose tissue beiging^{9,11,12,22}. To define the contribution of ILC2 and neuroregulatory cues in this process, the expression of the uncoupling protein 1 (UCP1) was assessed in RET loss- and gain-of-function models. When compared to their littermate controls, Ret^{ΔVav1} mice and Ret^{ΔIl5} mice had reduced Ucp1, Cox8b, and Cidea expression in the GAT, while Ret^{MEN2B} BM chimaeras displayed increased Ucp1 levels (FIGS. 3N, 3O, 8D). Further evidence that ILC2-autonomous GDNF-RET cues are required for Ucp1 expression was provided by ILC2 complementation of GAT explant cultures from Rag1^{-/-}Il2rg^{-/-} mice (FIG. 3P, 8E). Notably, addition of GDNF to GAT explant/ILC2 co-cultures efficiently induced Ucp1 expression in a RET dependent manner (FIGS. 3Q, 8E). In aggregate, these data indicate that ILC2-intrinsic neurotrophic factor cues are required to control adipose homeostasis and obesity.

### Example 4: A Novel Aorticorenal-Adipose Circuit that Connects to Discrete Brain Areas and Controls GAT ILC2 Function

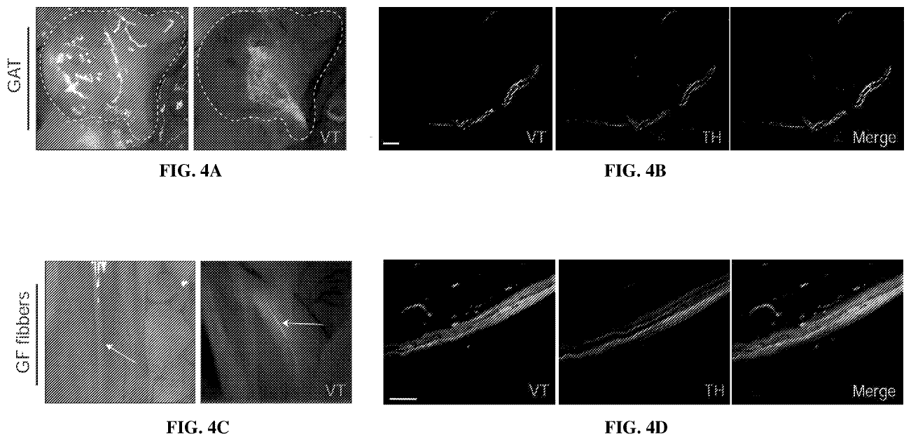

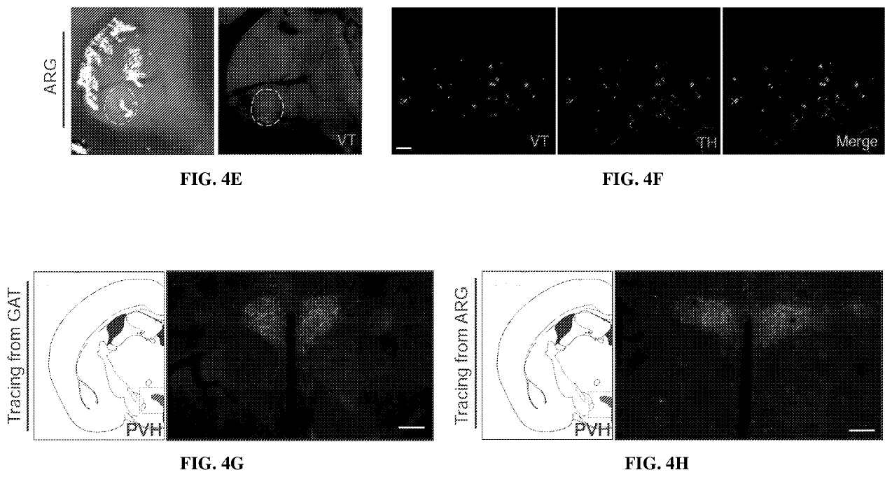

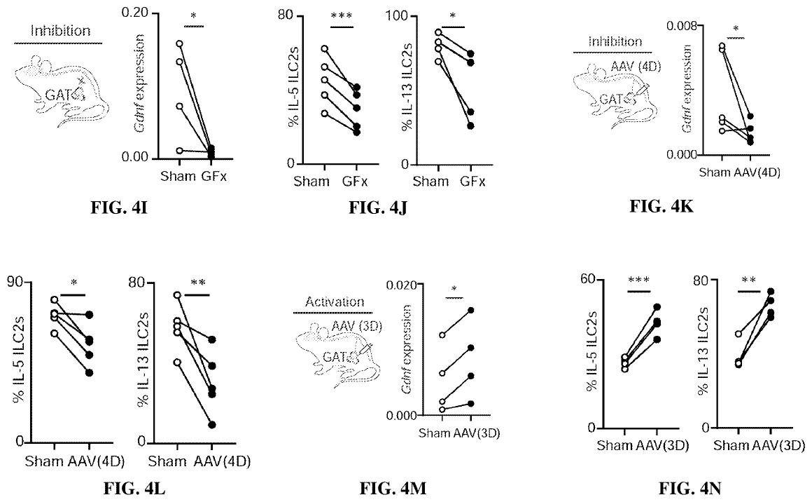

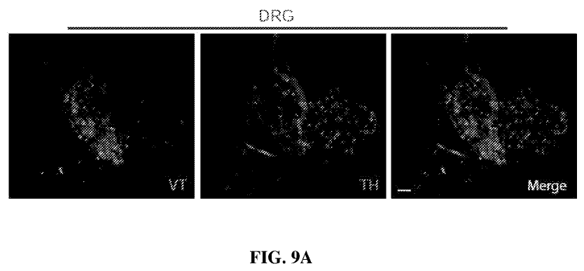

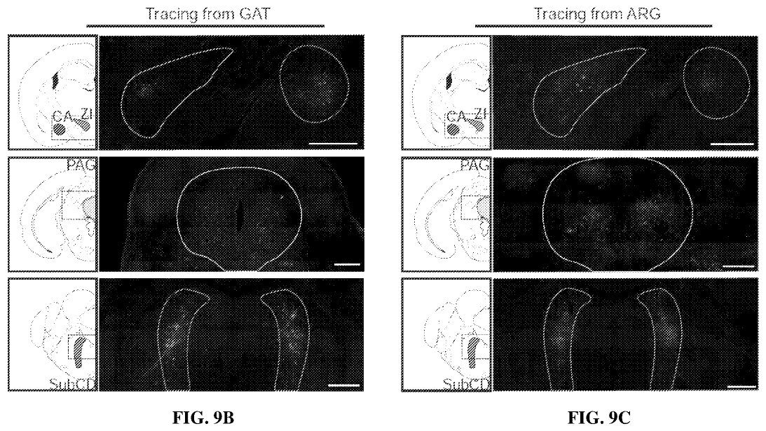

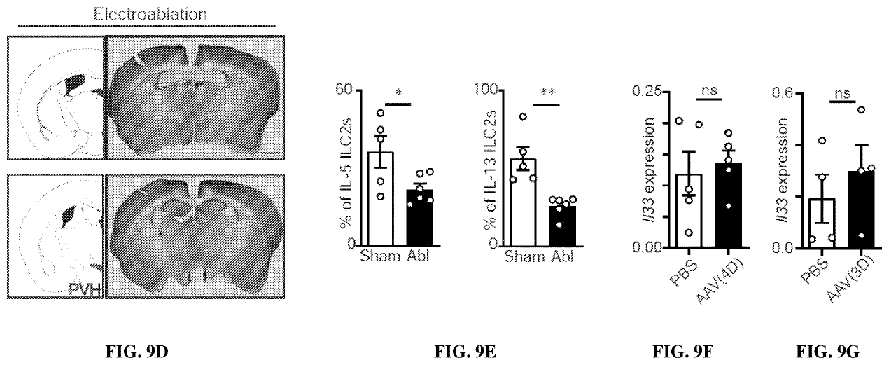

To examine how local sympathetic fibers integrate regional and higher-order circuits, the connections of GAT neurons were characterized. Initially, viral tracing (VT) was performed by injecting retrograde green fluorescent protein (GFP)-labelled adeno-associated virus (AAV) into the GAT. Analysis of such mice revealed the infection of tyrosine hydroxylase (TH) positive neurons in the adipose tissue and sympathetic TH positive fibers of the genitofemoral nerve running longitudinally to the ventral side of the psoas muscle (FIGS. 4A-4D). Importantly, discrete neuronal cell bodies were traced to the prevertebral aorticorenal ganglion (ARG) and dorsal root ganglia (DRG) (FIGS. 4E, 4F, 9A).

Nevertheless, while GFP labelled cell bodies in the aorticorenal ganglia were dopaminergic, their DRG counterparts were TH negative, indicating that the renal ganglion harbours cell bodies of the efferent sympathetic innervation of the GAT (FIGS. 4E, 4F, 9A). To interrogate if the GAT-aorticorenal axis connects to high-order circuits, polysynaptic tracing was performed using fluorescent protein-producing pseudorabies virus (PRV). Retrograde tracing with PRV from the GAT or from the aorticorenal ganglion revealed polysynaptic connections to overlapping discrete brain areas in the brain stem, mid-brain, amygdala and hypothalamus (FIGS. 4G, 4H, 9B, 9C). Noteworthy, the paraventricular nucleus of the hypothalamus (PVH) was consistently traced from the GAT and from the aorticorenal ganglion, indicating that the GAT connects polysynaptically to this hypothalamic nucleus (FIGS. 4G, 4H). Interestingly, the PVH was previously reported to regulate the brain sympathetic outflow to peripheral body tissues^{23,24}, which is consistent with impaired adipose innate type 2 cytokines observed in mice with surgical stereotaxic PVH ablation (FIGS. 9D, 9E).

To further dissect the impact of the aorticorenal neuronal circuit on GAT ILC2, unilateral surgical ablations were performed on the genitofemoral nerve (GFx). When compared to the sham contralateral controls, the GFx GAT harboured ILC2 with impaired function that associated with decreased GDNF expression (FIGS. 41, 4J). To better define the link between GAT innervating sympathetic aorticorenal neurons and ILC2 function, the activity of these neurons was modulated using chemogenetic approaches. Thus, inhibitor or activator DREADD-carrying adeno-associated virus (AAV) (AAV(4D) and AAV(3D), respectively) were injected unilaterally in the GAT. Sequentially, the designer drug (Clozapine-N-oxide (CNO)) that leads to neuronal inhibition or stimulation of DREADD-carrying neurons was injected. When compared to their respective sham contralateral controls, inhibition of AAV(4D)-expressing neurons led to reduced GDNF and innate type 2 cytokines, while activations of AAV(3D)-carrying neurons resulted in increased GDNF and ILC2 function (FIGS. 4K-4N). In contrast, inhibition of AAV(4D)-expressing neurons and activation of AAV (3)D-expressing neurons did not affect interleukin-33 (Il33) expression (FIGS. 9F, 9G). Taken together, these data reveal a novel aorticorenal-adipose circuit that connects to discrete brain areas and controls GAT ILC2 function.

### Example 5: Discussion

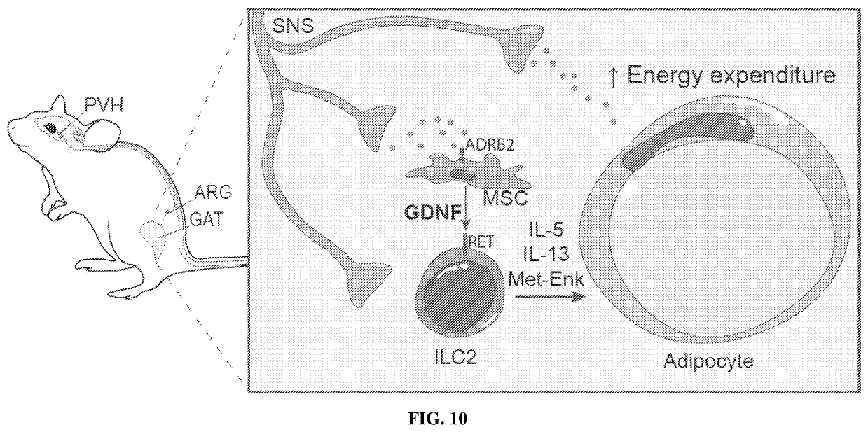

Defining whether neuronal circuits and immune cells cooperate to drive inter-organ communication is critical to understand organismic physiology and systemic diseases. This work establishes an unappreciated inter-organ and multi-tissue communication circuitry that integrates neuronal- and mesenchymal-derived signals to orchestrate ILC2 function and obesity. There is a brain-body axis conveys to a sympathetic aorticorenal-adipose interface that regulates ILC2. Notably, neuro-mesenchyme units translate the sympathetic tone into neurotrophic factor expression in the GAT. In turn, neurotrophic factors control adipose ILC2 function via the neuroregulatory receptor RET, shaping the host metabolism, energy expenditure and obesity (FIG. 10).

Adipose mesenchymal cells were shown to regulate local immune cell homeostasis via the expression of IL-33²⁵⁻²⁷. Here, it was demonstrated that mesenchymal cells link neuronal cues to adipose ILC2 function, via the production of GDNF. While sympathetic cues were shown to directly inhibit pulmonary ILC2 during infection²⁸, adrenergic signals indirectly activate GAT ILC2, indicating that sympathetic signals may encompass dual mechanisms to activate or repress ILC2 in a context- and organ-dependent manner.

Neuronal sympathetic cues directly mediate fat breakdown in the context of neuro-adipose connections⁷. This work indicates that sympathetic cues indirectly regulate energy expenditure via neuro-mesenchyme interactions that lead to ILC2-derived cytokine production. As such, coupling these direct and indirect sympathetic effects to regulate energy homeostasis may have ensured efficient and integrated multi-tissue responses to dietary challenges. Given the importance of the brain PVH in integrating systemic metabolic cues²⁴, in modulating sympathetic outflow^{23,24}, and its connection to the aorticorenal-adipose circuit (FIGS. 4, 10), it is tempting to hypothesise that the PVH acts as a central hub that translates metabolic body states into peripheral immune functions that ensure energy homeostasis.

Finally, this data may also grant better knowledge on how abnormal neuronal and immune functions associate with obesity and metabolic disorders in humans^{9,29,30}.

### Example 6: Materials & Methods and References

### Materials and Methods

Mice: C57BL/6J mice were purchased from Charles River and bred with C57BL/6J Ly5.1 in order to obtain C57BL/6 Ly5.1/Ly5.2 (CD45.1/CD45.2). Gfap-Cre³¹, Pdgfra-Cre³², Vav1-Cre³³, Il7ra-Cre³⁴, Il5-Cre³⁵, Th-Cre³⁶, Adrb2^{fl/fl 37}, Rag1^{-/- 38}, Il2rg^{-/- 39}, Ret^{MEN2B 40}, ROSA26·RFP⁴¹, ROSA26·3D⁴², ROSA26·DTR⁴³, Ret^{fl/fl 44}, Gfra1^{-/- 45}, Gfra2^{-/- 46}, and Gfra3^{-/- 47}, were in a full C57BL/6J background. Mice were bred and maintained at the Champalimaud Centre for the Unknown (CCU) animal facilities under specific pathogen free conditions. Mice were systematically compared with co-housed littermate controls unless stated otherwise. 8-9-week-old females were used in this study. Power analysis was performed to estimate the number of experimental mice. All animal experiments were approved by national and institutional ethical committees, respectively, Direção Geral de Veterinária and CCU ethical committees. Randomisation and blinding were not used unless stated otherwise.

Cell isolation: For the isolation of adipose tissue cells, tissue was collected into PBS, cut into small pieces and incubated with Liberase TM (2.5 μg/ml, Roche) and DNase I (20 U/ml; Roche) for 1 h at 37° C. under gentle agitation. A single cell suspension was obtained by passage through a 100 μm cell strainer (Thermo Fisher Scientific) and centrifugation was used to separate the stromal vascular fraction from the adipocyte fraction. Erythrocytes were lysed with red blood cell lysis buffer (eBioscience) and removed by centrifugation.

Flow cytometry and cell sorting: For cytokine analysis ex vivo, cells were incubated with PMA (50 ng/ml), ionomycin (500 ng/ml) (Sigma) and brefeldin A (eBioscience) in complete RPMI (supplemented with 10% foetal bovine serum (FBS), 1% HEPES, sodium pyruvate, glutamine, streptomycin and penicillin (Corning)) for 4 hours prior to intracellular staining, unless stated otherwise. Intracellular staining was performed using IC fixation/permeabilization kit (eBioscience). Cell suspensions were stained with anti-CD45 (30-F11; 1:200); anti-CD45.1 (A20; 1:200); anti-CD45.2 (104; 1:200); anti-CD11c (N418; 1:200); anti-CD11b (Mi/70; 1:400); anti-CD8a (53-6.7; 1:200); anti-CD19 (eBiolD3; 1:200); anti-NK1.1 (PK136; 1:100); anti-CD3c (eBio500A2; 1:200); anti-TER119 (TER-119; 1:200); anti-Gr1 (RB6-8C5; 1:400); anti-CD4 (RM4-5; 1:200); anti-CD90.2 (Thy1.2; 53-2.1; 1:200); anti-TCRβ (H57-595; 1:200); anti-TCRγδ (GL3; 1:200); anti-B220 (RA3-6B2; 1:200); anti-KLRG1 (2F1/KLRG1; 1:200); antiLy-6A/E (Sca1; D7; 1:200); anti-CD16/CD32 (93; 1:50); anti-gp38 (eBio8.1.1; 1:100); anti-F4/80 (BM8; 1:200); anti-IL-4 (11B11; 1:100) from eBiosciences. Anti-PDGFRA (APA5; 1:400) and anti-CD31 (MEC13.3; 1:200) from Biolegend. Anti-IL-5 (TRFK5; 1:200) from BD Biosciences. Anti-GDNF (B-8; 1:200) was purchased from Santa Cruz biotechnology. Anti-Met-Enk (bs-1759R-A680; 1:400) from Bioss. LIVE/DEAD Fixable Aqua Dead Cell Stain Kit (1:50) and anti-IL-13 (eBiol3A; 1:200) were purchased from Invitrogen. Cell populations were gated on live cells and defined as ILC2: CD45⁺Lin⁻Thy1.2⁺Sca⁻1+KLRG1+, lineage was composed by CD3ε, CD8α, TCRβ, TCRγδ, CD19, Gr1, CD11c, CD11b and TER119; glial cells: CD45⁻CD31⁻GFAP⁺, MSCs: CD45⁻CD31⁻PDGFRA⁺gp38⁺, endothelial cells: CD45⁻CD31⁺. Flow cytometry analysis and cell sorting were performed using FACSFusion, LSRFortessa and LSRFortessa X-20 (BD Biosciences). Sorted populations were >95% pure. Data analysis was done using FlowJo v10 software (Tristar).

Sympathetic manipulation: Chemical sympathetic ablation was performed by injecting 200 mg/kg 6-OHDA (Sigma) intraperitonially, 3 days and 1 day before analysis. Control mice were injected on the same days with the PBS 0.4% ascorbic acid (Sigma) used as a vehicle for 6-OHDA. Sympathetic ablation was also performed by administering pegylated diphtheria toxin to R26/DTR^{Th} mice as previously described¹⁴. For activation of ADRB2, its agonist clenbuterol (Sigma) was administered in the drinking water with 4% sucrose to a final concentration of 10 mg/kg/day, for 8 days. Control animals were given water with 4% sucrose for 8 days. For chemogenetic activation of sympathetic neurons in R26/3D^{Th} mice, 4 mg/kg CNO (Sigma) were administered, 2 mg/kg in the drinking water with 4% sucrose, and 2 mg/kg intraperitonially in PBS⁴⁸.

Quantitative RT-PCR: Total RNA from sorted or cultured cells was extracted using RNeasy micro kit or RNeasy mini kit (Qiagen) according to the manufacturer's protocol. When indicated, total adipose tissue or adipocyte fraction was collected to Trizol (Invitrogen) followed by chloroform and isopropanol RNA extraction, according to the manufacturer's protocol. RNA concentration was determined using Nanodrop Spectrophotometer (Nanodrop Technologies). Quantitative real-time PCR was performed in StepOne and QuantStudio 5 real-time PCR systems (Applied Biosystems) with Hprt and Gapdh as housekeeping genes. Briefly, High Capacity RNA-to-cDNA Kit (Applied Biosystems) were used to retro-transcribe RNA, followed by a pre-amplification PCR using TaqMan PreAmp Master Mix (Applied Biosystems). TaqMan Gene Expression Master Mix (Applied Biosystems) was used in the real-time PCR. TaqMan Gene Expression Assays (Applied Biosystems) were the following: Hprt Mm00446968_m1; Gapdh Mm99999915_g1; 115 Mm00439646_m1; 1113 Mm00434204_m1; Areg Mm01354339_m1; Penk Mm01212875_m1; Ret Mm00436304_m1; Gdnf Mm00599849_m1; Ucp1 Mm01244861_m1; Adrb2 Mm02524224_s1; Il25 Mm00499822_m1; and Il33 Mm00505403_m1. Analysis was performed using the comparative CT method (2^{-ΔCT}). When comparison or fold change between samples was required, the comparative ΔCT method (2^{-ΔΔCT}) was applied.

RNA sequencing and data analysis: PDGFRA+MSCs from mice treated with 6-OHDA or vehicle were isolated, and RNA was extracted and purified, as previously described. RNA quality was assessed using an Agilent 2100 Bioanalyzer (Agilent Technologies). Sequencing was performed on an HiSeq4000 platform (PE100, Illumina). Global quality of FASTQ files with raw RNA-sequencing reads was analysed using FastQC (version 0.11.9)⁴⁹. Trimmomatic-0.39 was used to remove the first 10 base pairs (HEADCROP=10)⁵⁰. Aligning and read processing were performed using STAR 2.7.3a (https://github.com/alexdobin/STAR/releases)⁵¹, sequences were aligned to the reference file of Mus musculus genome assembly GRCm38.p6 and the corresponding genome annotation file (http://www.ensembl.org/Mus_musculus/Info/Index). Volcano plot of differentially expressed genes was obtained using EdgeR (version 3.30.3) (https://bioconductor.org/packages/release/bioc/html/edgeR.html)⁵². DeSeq2 (version 1.28.1) (https://bioconductor.org/packages/release/bioc/html/DESeq2.html) was used to obtain the statistics of all genes⁵³. Genes with lower than 10 average reads, false discovery rate (FDR) above 0.05, and a log 2 (difference between groups) above −2 and below 2, were excluded from further analysis. A list of differentially expressed genes was thus obtained comprising 227 upregulated genes and 35 downregulated genes, in the 6-OHDA group. The heat maps of modulated genes were obtained by plotting the z-scores (normalized read counts per gene) using the GraphPad Prism software (GraphPad Software, La Jolla, Calif).

In vitro and in vivo MSC activation: For in vitro experiments, purified GAT PDGFRA+MSCs were cultured in complete DMEM (supplemented with 10% FBS, 1% HEPES, sodium pyruvate, glutamine, streptomycin and penicillin (Corning)) at 37° C. After 2 hours of rest in complete DMEM without FBS, MSCs were stimulated for 16 hours with 10 μg/ml clenbuterol. For RNA analysis, MSCs were lysed using RLT buffer (Qiagen). For GDNF protein analysis, MSCs were incubated with brefeldin A (eBioscience) during clenbuterol stimulation prior to intracellular staining.

In vitro and in vivo ILC2 activation: For in vitro experiments, purified GAT ILC2 were cultured in complete RPMI at 37° C. After 2 hours of rest in RPMI without FBS, ILC2 were stimulated for 3 hours with 50 ng/ml of GDNF family ligands (R&D Systems). For RNA analysis, ILC2 were lysed using RLT buffer (Qiagen). For cytokine protein analysis ex vivo, stimulated ILC2 were incubated with PMA (50 ng/mL), ionomycin (500 ng/mL) (Sigma) and brefeldin A (eBioscience) for 4 hours prior to intracellular staining.

Bone marrow and foetal liver chimeras: Bone marrow cells extracted from femurs and tibiae of Rag1^{-/-}·Ret^{MEN2B}, and Ret^{ΔVav1} mice and their respective littermate controls. Foetal livers were obtained from E13.0 Gfra1^{-/-} mice and their respective littermate controls. Bone marrow and foetal liver cells were CD3-depleted using Dynabeads Biotin Binder (Invitrogen) according to the manufacturer's instructions. 10⁶ cells of each genotype (CD45.2) were injected intravenously alone or in direct competition with a third-party WT competitor (CD45.1/CD45.2), in a 1:1 ratio, into non-lethally irradiated (3Gy) Rag1^{-/-}Il2rg^{-/-}mice (CD45.1). Mice were analysed at 10-12 weeks after transplantation.

High fat diet: Animals were placed on HFD (60Kj % fat (Lard) E15742-3407, Ssniff GmbH) for 16 weeks, unless stated otherwise. Glucose tolerance test was performed at 14 weeks after the start of HFD administration. Glucose (Sigma) in PBS was administered at 2 mg/kg in mice fasted for 8 hours and glucose was measured using an ACCU-CHECK Aviva glucometer (Roche).

ILC2 adoptive transfer: ILC2 from Ret^{MEN2B}, Ret^{D} mice and their respective littermate controls were purified from visceral adipose tissue for adoptive transfer. Purified ILC2 were expanded in vitro in supplemented RPMI in the presence of recombinant mouse IL-2, IL-7 (10 ng/mL; Peprotech) and IL-33 (10 ng/mL; R&D Systems) for 8 days. 2×10⁵ ILC2 were injected intraperitonially into Rag1^{-/-}Il2rg^{-/-} recipients. Mice were placed on HFD two weeks after adoptive transfer.

Explant cultures: GAT was obtained from Rag1^{-/-}Il2rg^{-/-}, cut into 2 mm pieces and incubated for 4 hours in complete RPMI at 37° C. Approximately 10⁴ isolated GAT ILC2 from Ret^{WT} or Ret^{Δ} mice were then co-cultured with GAT explants and 50 ng/mL GDNF family ligands for 16 hours. Explants were collected to Trizol (Invitrogen) and disrupted by sonication for RNA analysis.

Virus administration: Viral tracing experiments were performed using a Hamilton® syringe (Hamilton) by injecting 10 μl of pseudorabies virus (PRV)-614 (PRV-Bartha containing the CMV-mRFP reporter gene cassette inserted into the gG locus of the viral genome) or pAAV-Ef1a-mCherry-IRES-Cre (Addgene viral prep #55632-AAVrg) into the gonadal fat pads. Aorticorenal ganglion injections of 1 μl (PRV)-614-RFP were performed using a Nanoject III Programmable Nanoliter Injector (Drummond Scientific). Brains were collected 6 days post-PRV injection, after perfusion with PBS and 4% PFA for fixation and further processing. For AAV tracing, GAT and aorticorenal ganglion were collected 3 weeks post-injection and fixed in 4% PFA for further processing. For neuronal function manipulation, rAAV-PGA-hM3DqDREADD-GFP (AAV(3D)) or rAAV-PGA-hM4DqDREADD-GFP (AAV(4D)) (Addgene) were injected on one gonadal fat pad while the contralateral fat pad was injected with PBS and used as a contralateral control. 4 mg/kg CNO were administered as described above. For activation of local neurons CNO was administered 4 weeks post-injection; for inactivation of local neurons CNO was administered 6 weeks post-injection.

PVH electroablation: Bilateral ablation of the PVH was performed in 9-12-week-old C57BL/6J mice by electrolytic lesion using stereotaxic brain surgery, as described previously⁵⁴. Mice were kept under deep anaesthesia using a mixture of isoflurane and oxygen (1-3% isoflurane at 11/min). Surgeries were performed using a stereotaxic device (Kopf). After identification of the bregma, a hole was drilled through which the lesion electrode was inserted into the brain. Electrodes were made by isolating a 0.25 mm stainless steel insect pin with a heat shrink polyester tubing, except for 0.5 mm at the tip. The electrode tip was aimed at the paraventricular hypothalamic nucleus, −0.35 mm anterior to bregma, 0.25 mm lateral to the midline, and 5.8 mm ventral to the skull top (Paxinos Mouse Brain Atlas, Franklin 2001). Bilateral lesions were made by passing 0.75 mA current through the electrode for the duration of 3 seconds, in the left and right side separately. Sham-lesioned mice underwent the same procedure, but no current was passed through the electrode. After surgery animals were housed individually with food and water ad libitum and were allowed to recover for 1 week. Successful lesioned mice were selected based on histopathology analysis. Mice were analysed after 10-12 weeks.

Immunofluorescence and microscopy: Brains from animals injected with PRV-614-RFP were cut into 50 μm slices using a microtome, and mounted in Mowiol (Sigma). Brain images and H&E staining adipose tissue images were obtained in a Zeiss AxioScan Z1 slide scanner (20× Plan Apochromat dry 0.80 0.55 objective). Gonadal adipose tissue was obtained from R26/RFP^{DGfap}, R26/RFP^{DPdfra} and C57BL/6J mice. For ultramicroscopy imaging, whole gonadal fat pads were collected and for confocal imaging pieces of approximately 1×1 mm were obtained from tissue fixed with 4% PFA at 4° C. overnight. Samples were blocked and permeabilized with PBS containing 0.6% Triton X-100 (Sigma) and 2% BSA (Sigma) and incubated for 1-2 days at room temperature with the following antibodies: anti-TH (P40101; Pel-Freez); anti-GDNF (B-8; Santa-Cruz); or CD31 (390, FITC; Abeam). Alexa Fluor 568 goat anti-rabbit and Alexa Fluor 488 goat anti-rabbit (Invitrogen) were used as secondary antibodies overnight at room temperature. Clearing of the gonadal tissue was done after staining starting with dehydration in a sequence of ethanol solutions of increasing concentrations for 24h each (20%, 40%, 60%, 80% and 100%) followed by immersion in Ethyl cinnamate⁵⁵. For cleared whole tissue imaging, samples were mounted in Ethyl cinnamate and acquired in a LightSheet Zeiss Z.1 (Plan Apochromat 20x/1.0, 2.4 objective). For confocal imaging, samples were mounted in Mowiol and acquired on a Zeiss LSM710 confocal microscope using Pl-Apochromat 25x/0.8 M27 immersion objective and Pl-Apochromat 63x/1.4 oil immersion objectives. For viral tracing infection confirmation, a Leica M205 stereomicroscope coupled to a Leica DFC7000 T camera (Leica Microsystems, Wetzlar, Germany) was used. Images were processed using ImageJ 1.53 (NIH), Zen Blue 3.0 (Zeiss) and Imaris 9.6 (Oxford Instruments).

Statistics: Results are shown as mean±s.e.m. Statistical analysis was performed with GraphPad Prism software (GraphPad Software, La Jolla, Calif). Student t-test was performed on homoscedastic populations. Welch's corrected t-test was applied on samples with different variances, unless stated otherwise. Paired t-test was applied to contralateral control samples. Results were considered significant at P<0.05.

### REFERENCES

1. Godinho-Silva, C., Cardoso, F. & Veiga-Fernandes, H. Neuro-Immune Cell Units: A New Paradigm in Physiology. Annu Rev Immunol 37, 19-46 (2019).
2. Huh, J. R. & Veiga-Fernandes, H. Neuroimmune circuits in inter-organ communication. Nat Rev Immunol 20, 217-228 (2020).
3. Veiga-Fernandes, H. & Artis, D. Neuronal-immune system cross-talk in homeostasis. Science 359, 1465-1466 (2018).
4. Chu, C., Artis, D. & Chiu, I. M. Neuro-immune Interactions in the Tissues. Immunity 52, 464-474 (2020).
5. Bartness, T. J., Vaughan, C. H. & Song, C. K. Sympathetic and sensory innervation of brown adipose tissue. Int J Obes (Lond) 34 Suppl 1, S36-42 (2010).
6. Morrison, S. F. Central neural control of thermoregulation and brown adipose tissue. Auton Neurosci 196, 14-24 (2016).
7. Zeng, W. et al. Sympathetic neuro-adipose connections mediate leptin-driven lipolysis. Cell 163, 84-94 (2015).
8. Moro, K. et al. Innate production of TH2 cytokines by adipose tissue-associated c-Kit+Sca−1+lymphoid cells. Nature 463, 540-544 (2010).
9. Brestoff, J. R. et al. Group 2 innate lymphoid cells promote beiging of white adipose tissue and limit obesity. Nature 519, 242-246 (2015).
10. Hams, E. et al. The helminth T2 RNase omegal promotes metabolic homeostasis in an IL-33− and group 2 innate lymphoid cell-dependent mechanism. FASEB J 30, 824-835 (2016).
11. Lee, M. W. et al. Activated type 2 innate lymphoid cells regulate beige fat biogenesis. Cell 160, 74-87 (2015).
12. Molofsky, A. B. et al. Innate lymphoid type 2 cells sustain visceral adipose tissue eosinophils and alternatively activated macrophages. The Journal of experimental medicine 210, 535-549 (2013).
13. Vivier, E. et al. Innate Lymphoid Cells: 10 Years On. Cell 174, 1054-1066 (2018).
14. Pereira, M. M. et al. A brain-sparing diphtheria toxin for chemical genetic ablation of peripheral cell lineages. Nat Commun 8, 14967 (2017).
15. Arighi, E., Borrello, M. G. & Sariola, H. RET tyrosine kinase signaling in development and cancer. Cytokine Growth Factor Rev 16, 441-467 (2005).
16. Mulligan, L. M. RET revisited: expanding the oncogenic portfolio. Nat Rev Cancer 14, 173-186 (2014).
17. Fonseca-Pereira, D. et al. The neurotrophic factor receptor RET drives haematopoietic stem cell survival and function. Nature 514, 98-101 (2014).
18. Ibiza, S. et al. Glial-cell-derived neuroregulators control type 3 innate lymphoid cells and gut defence. Nature 535, 440-443 (2016).
19. Patel, A. et al. Differential RET signaling pathways drive development of the enteric lymphoid and nervous systems. Sci Signal 5, ra55 (2012).
20. Veiga-Fernandes, H. et al. Tyrosine kinase receptor RET is a key regulator of Peyer's Patch organogenesis. Nature 446, 547-551 (2007).
21. Kahn, B. B. & Flier, J. S. Obesity and insulin resistance. The Journal of clinical investigation 106, 473-481 (2000).
22. Qiu, Y. et al. Eosinophils and type 2 cytokine signaling in macrophages orchestrate development of functional beige fat. Cell 157, 1292-1308 (2014).
23. Morrison, S. F., Madden, C. J. & Tupone, D. Central neural regulation of brown adipose tissue thermogenesis and energy expenditure. Cell Metab 19, 741-756 (2014).
24. Sutton, A. K., Myers, M. G., Jr. & Olson, D. P. The Role of PVH Circuits in Leptin Action and Energy Balance. Annu Rev Physiol 78, 207-221 (2016).
25. Mahlakoiv, T. et al. Stromal cells maintain immune cell homeostasis in adipose tissue via production of interleukin-33. Sci Immunol 4 (2019).
26. Rana, B. M. J. et al. A stromal cell niche sustains ILC2-mediated type-2 conditioning in adipose tissue. The Journal of experimental medicine 216, 1999-2009 (2019).
27. Spallanzani, R. G. et al. Distinct immunocyte-promoting and adipocyte-generating stromal components coordinate adipose tissue immune and metabolic tenors. Sci Immunol 4 (2019).
28. Moriyama, S. et al. beta2-adrenergic receptor-mediated negative regulation of group 2 innate lymphoid cell responses. Science 359, 1056-1061 (2018).
29. Andersen, C. J., Murphy, K. E. & Fernandez, M. L. Impact of Obesity and Metabolic Syndrome on Immunity. Adv Nutr 7, 66-75 (2016).
30. Guilherme, A., Henriques, F., Bedard, A. H. & Czech, M. P. Molecular pathways linking adipose innervation to insulin action in obesity and diabetes mellitus. Nat Rev Endocrinol 15, 207-225 (2019).
31. Zhuo, L. et al. hGFAP-cre transgenic mice for manipulation of glial and neuronal function in vivo. Genesis 31, 85-94 (2001).
32. Roesch, K. et al. The transcriptome of retinal Muller glial cells. J Comp Neurol 509, 225-238 (2008).
33. de Boer, J. et al. Transgenic mice with hematopoietic and lymphoid specific expression of Cre. Eur J Immunol 33, 314-325 (2003).
34. Schlenner, S. M. et al. Fate mapping reveals separate origins of T cells and myeloid lineages in the thymus. Immunity 32, 426-436 (2010).
35. Nussbaum, J. C. et al. Type 2 innate lymphoid cells control eosinophil homeostasis. Nature 502, 245-248 (2013).
36. Savitt, J. M., Jang, S. S., Mu, W., Dawson, V. L. & Dawson, T. M. Bcl-x is required for proper development of the mouse substantia nigra. The Journal of neuroscience: the official journal of the Society for Neuroscience 25, 6721-6728 (2005).
37. Hinoi, E. et al. The sympathetic tone mediates leptin's inhibition of insulin secretion by modulating osteocalcin bioactivity. J Cell Biol 183, 1235-1242 (2008).
38. Mombaerts, P. et al. RAG-1-deficient mice have no mature B and T lymphocytes. Cell 68, 869-877. (1992).
39. Cao, X. et al. Defective lymphoid development in mice lacking expression of the common cytokine receptor gamma chain. Immunity 2, 223-238 (1995).
40. Smith-Hicks, C. L., Sizer, K. C., Powers, J. F., Tischler, A. S. & Costantini, F. C-cell hyperplasia, pheochromocytoma and sympathoadrenal malformation in a mouse model of multiple endocrine neoplasia type 2B. Embo J 19, 612-622 (2000).
41. Madisen, L. et al. A robust and high-throughput Cre reporting and characterization system for the whole mouse brain. Nature neuroscience 13, 133-140 (2010).
42. Sciolino, N. R. et al. Recombinase-Dependent Mouse Lines for Chemogenetic Activation of Genetically Defined Cell Types. Cell Rep 15, 2563-2573 (2016).
43. Buch, T. et al. A Cre-inducible diphtheria toxin receptor mediates cell lineage ablation after toxin administration. Nature methods 2, 419-426 (2005).
44. Almeida, A. R. et al. RET/GFRalpha signals are dispensable for thymic T cell development in vivo. PLoS One 7, e52949 (2012).
45. Enomoto, H. et al. GFR alpha1-deficient mice have deficits in the enteric nervous system and kidneys. Neuron 21, 317-324 (1998).
46. Rossi, J. et al. Retarded growth and deficits in the enteric and parasympathetic nervous system in mice lacking GFR alpha2, a functional neurturin receptor. Neuron 22, 243-252 (1999).
47. Nishino, J. et al. GFR alpha3, a component of the artemin receptor, is required for migration and survival of the superior cervical ganglion. Neuron 23, 725-736 (1999).
48. Zhu, H. & Roth, B. L. Silencing synapses with DREADDs. Neuron 82, 723-725 (2014).
49. Leggett, R. M., Ramirez-Gonzalez, R. H., Clavijo, B. J., Waite, D. & Davey, R. P. Sequencing quality assessment tools to enable data-driven informatics for high throughput genomics. Front Genet 4, 288 (2013).
50. Bolger, A. M., Lohse, M. & Usadel, B. Trimmomatic: a flexible trimmer for Illumina sequence data. Bioinformatics 30, 2114-2120 (2014).
51. Dobin, A. et al. STAR: ultrafast universal RNA-seq aligner. Bioinformatics 29, 15-21 (2013).
52. McCarthy, D. J., Chen, Y. & Smyth, G. K. Differential expression analysis of multifactor RNA-Seq experiments with respect to biological variation. Nucleic Acids Res 40, 4288-4297 (2012).
53. Love, M. I., Huber, W. & Anders, S. Moderated estimation of fold change and dispersion for RNA-seq data with DESeq2. Genome Biol 15, 550 (2014).
54. Godinho-Silva, C. et al. Light-entrained and brain-tuned circadian circuits regulate ILC3s and gut homeostasis. Nature 574, 254-258 (2019).
55. Huang, J. et al. A cationic near infrared fluorescent agent and ethyl-cinnamate tissue clearing protocol for vascular staining and imaging. Sci Rep 9, 521 (2019).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for increasing activity or proliferation of Group 2 innate lymphoid cells (ILC2s), comprising contacting ILC2s with a rearranged during transfection (RET) agonist and/or contacting mesenchymal stromal cells (MSCs) with a beta-2-adrenergic receptor (ADRB2) agonist.

2. The method of claim 1, wherein the RET agonist comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

3. The method of claim 2, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

4. The method of any one of claims 1-3, wherein the ADRB2 agonist is clenbuterol, bitolterol, fenoterol, isoproterenol, levalbuterol, metaproterenol, pirbuterol, procaterol, ritodrine, albuterol, terbutaline, aformoterol, bambuterol, formoterol, salmeterol, abediterol, carmoterol, indacaterol, olodaterol, vailanterol, isoxsuprine, mabuterol, zilpaterol, or a combination thereof.

5. The method of any one of claims 1-4, wherein the contacting is in vitro.

6. The method of any one of claims 1-4, wherein the contacting is in vivo.

7. The method of claim any one of claims 1-4 or 6, wherein the RET agonist and/or ADRB2 agonist is administered to a subject.

8. The method of claim 7, wherein the subject is a human.

9. The method of any one of claims 1-8, wherein the ILC2s and/or the MSCs are in adipose tissue or derived from adipose tissue.

10. The method of claim 9, wherein the adipose tissue is gonadal adipose tissue (GAT).

11. A method for increasing production of interleukin-5 (IL-5), interleukin-13 (IL-13), and/or Met-enkephalin (Met-Enk) by Group 2 innate lymphoid cells (ILC2s), comprising contacting adipose ILC2s with a rearranged during transfection (RET) agonist and/or contacting mesenchymal stromal cells (MSCs) with a beta-2-adrenergic receptor (ADRB2) agonist.

12. The method of claim 11, wherein the RET agonist comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

13. The method of claim 12, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

14. The method of any one of claims 11-13, wherein the ADRB2 agonist is clenbuterol, bitolterol, fenoterol, isoproterenol, levalbuterol, metaproterenol, pirbuterol, procaterol, ritodrine, albuterol, terbutaline, aformoterol, bambuterol, formoterol, salmeterol, abediterol, carmoterol, indacaterol, olodaterol, vailanterol, isoxsuprine, mabuterol, zilpaterol, or a combination thereof.

15. The method of any one of claims 11-14, wherein the contacting is in vitro.

16. The method of any one of claims 11-14, wherein the contacting is in vivo.

17. The method of claim any one of claims 11-14 or 16, wherein the RET agonist and/or ADRB2 agonist is administered to a subject.

18. The method of claim 17, wherein the subject is a human.

19. The method of any one of claims 11-18, wherein the ILC2s and/or the MSCs are in adipose tissue or derived from adipose tissue.

20. The method of claim 19, wherein the adipose tissue is gonadal adipose tissue (GAT).

21. A method for decreasing susceptibility to obesity and/or increasing adipose homeostasis, comprising
(a) administering to a subject a rearranged during transfection (RET) agonist that contacts Group 2 innate lymphoid cells (ILC2s) in adipose tissue,
(b) administering to the subject a beta-2 adrenergic receptor (ADRB2) agonist that contacts mesenchymal stromal cells (MSCs) in adipose tissue, or
(c) a combination thereof.

22. The method of claim 21, wherein increased adipose homeostasis is increased glucose tolerance and/or decreased gonadal adipose tissue (GAT) fat mass.

23. The method of claim 21 or claim 22, wherein the RET agonist comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

24. The method of claim 23, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

25. The method of any one of claims 21-24, wherein the ADRB2 agonist is clenbuterol, bitolterol, fenoterol, isoproterenol, levalbuterol, metaproterenol, pirbuterol, procaterol, ritodrine, albuterol, terbutaline, aformoterol, bambuterol, formoterol, salmeterol, abediterol, carmoterol, indacaterol, olodaterol, vailanterol, isoxsuprine, mabuterol, zilpaterol, or a combination thereof.

26. The method of any one of claims 21-25, wherein the subject is a human.

27. A method of treating a disorder associated with decreased Group 2 innate lymphoid cell (ILC2) activity or proliferation, comprising
(a) administering to a subject a rearranged during transfection (RET) agonist that contacts ILC2s in adipose tissue,
(b) administering to the subject a beta-2 adrenergic receptor (ADRB2) agonist that contacts mesenchymal stromal cells (MSCs) in adipose tissue, or
(c) a combination thereof.

28. The method of claim 27, wherein the disorder is weight gain, obesity, diabetes, metabolic syndrome, or a combination thereof.

29. The method of claim 27 or claim 28, wherein the RET agonist comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

30. The method of claim 29, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

31. The method of any one of claims 27-30, wherein the ADRB2 agonist is clenbuterol, bitolterol, fenoterol, isoproterenol, levalbuterol, metaproterenol, pirbuterol, procaterol, ritodrine, albuterol, terbutaline, aformoterol, bambuterol, formoterol, salmeterol, abediterol, carmoterol, indacaterol, olodaterol, vailanterol, isoxsuprine, mabuterol, zilpaterol, or a combination thereof.

32. The method of any one of claims 27-31, wherein the subject is a human.

33. A method of treating a disorder associated with increased Group 2 innate lymphoid cell (ILC2) activity or proliferation, comprising
(a) administering to a subject a rearranged during transfection (RET) antagonist that contacts ILC2s in adipose tissue,
(b) administering to a subject a beta-2 adrenergic receptor (ADRB2) antagonist that contacts mesenchymal stromal cells (MSCs) in adipose tissue, or
(c) a combination of (a) and (b).

34. The method of claim 33, wherein the disorder is hypothermia, cachexia, allergy, helminth infection, allergic asthma, atopic dermatitis, intestinal inflammatory disease, or a combination thereof.

35. The method of claim 33 or claim 34, wherein the RET antagonist is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib.

36. The method of claim 35, wherein the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN).

37. The method of claim 35, wherein the inhibitory nucleic acid molecule is a sRNA, shRNA, or antisense nucleic acid molecule.

38. The method of any one of claims 33-37, wherein the ADRB2 antagonist is butoxamine, ICI-118,551, propranolol, oxprenolol, penbutolol, pindolol, sotalol, timolol, bucindolol, carteolol, carvedilol, labetalol, nadolol, or a combination thereof.

39. The method of any one of claims 33-38, wherein the subject is a human.

40. A method of treating cold exposure, comprising
(a) administering to a subject a rearranged during transfection (RET) agonist that contacts Group 2 innate lymphoid cells (ILC2s),
(b) administering to the subject a beta-2 adrenergic receptor (ADRB2) agonist that contacts ILC2s, or
(c) a combination thereof.

41. The method of claim 40, wherein the RET agonist comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

42. The method of claim 41, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

43. The method of any one of claims 40-42, wherein the ADRB2 agonist is clenbuterol, bitolterol, fenoterol, isoproterenol, levalbuterol, metaproterenol, pirbuterol, procaterol, ritodrine, albuterol, terbutaline, aformoterol, bambuterol, formoterol, salmeterol, abediterol, carmoterol, indacaterol, olodaterol, vailanterol, isoxsuprine, mabuterol, zilpaterol, or a combination thereof.

44. The method of any one of claims 40-43, wherein the subject is a human.

45. The method of any one of claims 40-44, wherein administering the RET agonist and/or the ADRB2 agonist increases the body temperature of the subject.
